(19) United States
(12) Patent Application Publication
(10) Pub. No.: US 2016/0031863 A1
(43) Pub. Date: Feb. 4, 2016

(54) HISTONE DEACETYLASE INHIBITORS AND COMPOSITIONS AND METHODS OF USE THEREOF

(71) Applicant: CHDI FOUNDATION, INC., New York, NY (US)

(72) Inventors: Celia Dominguez, Los Angeles, CA (US); Ignacio Muñoz-Sanjuán, Los Angeles, CA (US); Michel Maillard, New York, NY (US); Huw D. Vater, Saffron Walden, Essex (GB); Rebecca E. Jarvis, Saffron Walden (GB); Christopher A. Luckhurst, Cambridge (GB); Roland W. Bürli, Hertfordshire (GB); Grant Wishart, Saffron Walden (GB); Andrew J. Stott, Saffron Walden, Essex (GB)

(21) Appl. No.: 14/776,058

(22) PCT Filed: Mar. 10, 2014
(86) PCT No.: PCT/US14/22550

§ 371 Date: Sep. 14, 2015

Related U.S. Application Data
(60) Provisional application No. 61/785,551, filed on Mar. 14, 2013.

(51) Int. Cl. C07D 409/04; C07D 207/34; C07D 231/14; C07D 401/04; C07D 403/04

(52) U.S. Cl. CPC C07D 409/04; C07D 401/04; C07D 403/04; C07D 231/14; C07D 207/34

(57) ABSTRACT

Provided are certain histone deacetylase (HDAC) inhibitors of Formula I, compositions thereof, and methods of their use.

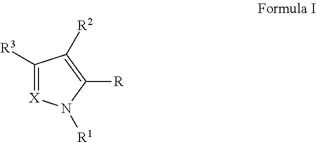

This application claims the benefit of priority of U.S. Provisional application No. 61/785,551, filed Mar. 14, 2013, which is incorporated by reference in its entirety.

Provided herein are certain histone deacetylase (HDAC) inhibitors, compositions thereof, and methods of their use.

Histone deacetylases (HDACs) are zinc-containing enzymes which catalyse the removal of acetyl groups from the ε-amino termini of lysine residues clustered near the amino terminus of nucleosomal histones. There are 11 known metal-dependent human histone deacetylases, grouped into four classes based on the structure of their accessory domains. Class I includes HDAC1, HDAC2, HDAC3, and HDAC8 and have homology to yeast RPD3. HDAC4, HDAC5, HDAC7, and HDAC9 belong to Class IIa and have homology to yeast HDAC1. HDAC6 and HDAC10 contain two catalytic sites and are classified as Class IIb, whereas HDAC11 has conserved residues in its catalytic center that are shared by both Class I and Class II deacetylases and is sometimes placed in Class IV.

Provided is a compound of Formula I

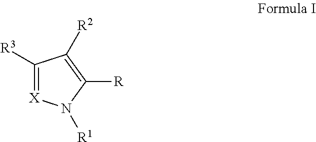

or a pharmaceutically acceptable salt thereof, wherein
X is CR⁴ or N;
R is chosen from —C(O)NH(OH) and —N(OH)C(O)R⁷;
R¹ is optionally substituted aryl or optionally substituted heteroaryl;
R² is chosen from hydrogen, C₁-C₄ alkyl, halo, C₁-C₄ haloalkyl, and nitrile;
R³ is chosen from —OR⁵, —NR⁵R⁶, optionally substituted alkyl, optionally substituted aralkyl, optionally substituted aryl, optionally substituted heteroaryl, optionally substituted heterocycloalkyl, optionally substituted heterocycloalkenyl, optionally substituted cycloalkenyl and optionally substituted cycloalkyl;
R⁴ is chosen from hydrogen, halo, C₁-C₄ alkyl or C₁-C₄ haloalkyl;
R⁵ and R⁶ are independently chosen from hydrogen, optionally substituted C₁-C₄ alkyl, optionally substituted C₁-C₄ haloalkyl, optionally substituted aryl, optionally substituted heteroaryl, optionally substituted heterocycloalkyl, optionally substituted cycloalkyl, optionally substituted aralkyl and optionally substituted heteroaralkyl; or R⁵ and R⁶, together with the nitrogen atom to which they are attached, form an optionally substituted heterocycloalkyl; and
R⁷ is chosen from hydrogen, C₁-C₄ alkyl and C₁-C₄ haloalkyl.

Also provided is a pharmaceutical composition comprising a compound, or a pharmaceutically acceptable salt thereof, described herein and at least one pharmaceutically acceptable excipient. Also provided is a method of preparing a pharmaceutical composition comprising a compound, or a pharmaceutically acceptable salt thereof, described herein and at least one pharmaceutically acceptable excipient.

Also provided is a method of treating a condition or disorder mediated by at least one histone deacetylase in a subject in need of such a treatment which method comprises administering to the subject a therapeutically effective amount of a compound, or a pharmaceutically acceptable salt thereof, described herein.

As used in the present specification, the following words, phrases and symbols are generally intended to have the meanings as set forth below, except to the extent that the context in which they are used indicates otherwise.

A dash (“-”) that is not between two letters or symbols is used to indicate a point of attachment for a substituent. For example, —CONH₂ is attached through the carbon atom.

By “optional” or “optionally” is meant that the subsequently described event or circumstance may or may not occur, and that the description includes instances where the event or circumstance occurs and instances in which it does not. For example, “optionally substituted alkyl” encompasses both “alkyl” and “substituted alkyl” as defined below. It will be understood by those skilled in the art, with respect to any group containing one or more substituents, that such groups are not intended to introduce any substitution or substitution patterns that are sterically impractical, synthetically non-feasible and/or inherently unstable.

“Alkyl” encompasses straight chain and branched chain having, for example, the indicated number of carbon atoms, usually from 1 to 20 carbon atoms, for example 1 to 8 carbon atoms, such as 1 to 6 carbon atoms. For example C₁-C₆ alkyl encompasses both straight and branched chain alkyl of from 1 to 6 carbon atoms. Examples of alkyl groups include methyl, ethyl, propyl, isopropyl, n-butyl, sec-butyl, tert-butyl, pentyl, 2-pentyl, isopentyl, neopentyl, hexyl, 2-hexyl, 3-hexyl, 3-methylpentyl, and the like. Alkylene is another subset of alkyl, referring to the same residues as alkyl, but having two points of attachment. Alkylene groups will usually have from 2 to 20 carbon atoms, for example 2 to 8 carbon atoms, such as from 2 to 6 carbon atoms. For example, C₀ alkylene indicates a covalent bond and C₁ alkylene is a methylene group. When an alkyl residue having a specific number of carbons is named, all geometric isomers having that number of carbons are intended to be encompassed; thus, for example, “butyl” is meant to include n-butyl, sec-butyl, isobutyl and t-butyl; “propyl” includes n-propyl and isopropyl.

“Cycloalkyl” indicates a non-aromatic, fully saturated carbocyclic ring having, for example, the indicated number of carbon atoms, for example, 3 to 10, or 3 to 8, or 3 to 6 ring carbon atoms. Cycloalkyl groups may be monocyclic or polycyclic (e.g., bicyclic, tricyclic). Examples of cycloalkyl groups include cyclopropyl, cyclobutyl, cyclopentyl, cyclopentenyl and cyclohexyl, as well as bridged and caged ring groups (e.g., norbornane, bicyclo[2.2.2]octane). In addition, one ring of a polycyclic cycloalkyl group may be aromatic, provided the polycyclic cycloalkyl group is bound to the parent structure via a non-aromatic carbon. For example, a 1,2,3,4-tetrahydronaphthalen-1-yl group (wherein the moiety is bound to the parent structure via a non-aromatic carbon atom) is a cycloalkyl group, while 1,2,3,4-tetrahydronaphthalen-5-yl (wherein the moiety is bound to the parent structure via an aromatic carbon atom) is not considered a cycloalkyl group.

“Cycloalkenyl” indicates a non-aromatic ring having 3 to 10, or 3 to 8, or 3 to 6 ring carbon atoms, and at least one double bond derived by the removal of one molecule of hydrogen from two adjacent carbon atoms of the corresponding cycloalkyl.

By “alkoxy” is meant an alkyl group, for example, of the indicated number of carbon atoms attached through an oxygen bridge such as, for example, methoxy, ethoxy, propoxy, isopropoxy, n-butoxy, sec-butoxy, tert-butoxy, pentoxy, 2-pentyloxy, isopentoxy, neopentoxy, hexoxy, 2-hexoxy, 3-hexoxy, 3-methylpentoxy, and the like. Alkoxy groups will usually have from 1 to 6 carbon atoms attached through the oxygen bridge.

“Aryl” indicates an aromatic carbon ring having, for example, the indicated number of carbon atoms, for example, 6 to 12 or 6 to 10 carbon atoms. Aryl groups may be monocyclic or polycyclic (e.g., bicyclic, tricyclic). In some instances, both rings of a polycyclic aryl group are aromatic (e.g., naphthyl). In other instances, polycyclic aryl groups may include a non-aromatic ring (e.g., cycloalkyl, cycloalkenyl, heterocycloalkyl, heterocycloalkenyl) fused to an aromatic ring, provided the polycyclic aryl group is bound to the parent structure via an atom in the aromatic ring. Thus, a 1,2,3,4-tetrahydronaphthalen-5-yl group (wherein the moiety is bound to the parent structure via an aromatic carbon atom) is considered an aryl group, while 1,2,3,4-tetrahydronaphthalen-1-yl (wherein the moiety is bound to the parent structure via a non-aromatic carbon atom) is not considered an aryl group. Similarly, a 1,2,3,4-tetrahydroquinolin-8-yl group (wherein the moiety is bound to the parent structure via an aromatic carbon atom) is considered an aryl group, while 1,2,3,4-tetrahydroquinolin-1-yl group (wherein the moiety is bound to the parent structure via a non-aromatic nitrogen atom) is not considered an aryl group. However, the term “aryl” does not encompass or overlap with “heteroaryl”, as defined herein, regardless of the point of attachment (e.g., both quinolin-5-yl and quinolin-2-yl are heteroaryl groups). In some instances, aryl is phenyl or naphthyl. In certain instances, aryl is phenyl.

Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene.

The term “halo” includes fluoro, chloro, bromo, and iodo, and the term “halogen” includes fluorine, chlorine, bromine, and iodine.

“Heteroaryl” indicates an aromatic ring containing, for example, the indicated number of atoms (e.g., 5 to 12, or 5 to 10 membered heteroaryl) made up of one or more heteroatoms (e.g., 1, 2, 3 or 4 heteroatoms) selected from N, O and S and with the remaining ring atoms being carbon. Heteroaryl groups do not contain adjacent S and O atoms. In some embodiments, the total number of S and O atoms in the heteroaryl group is not more than 2. In some embodiments, the total number of S and O atoms in the heteroaryl group is not more than 1. Unless otherwise indicated, heteroaryl groups may be bound to the parent structure by a carbon or nitrogen atom, as valency permits. For example, “pyridyl” includes 2-pyridyl, 3-pyridyl and 4-pyridyl groups, and “pyrrolyl” includes 1-pyrrolyl, 2-pyrrolyl and 3-pyrrolyl groups. When nitrogen is present in a heteroaryl ring, it may, where the nature of the adjacent atoms and groups permits, exist in an oxidized state (i.e., N⁺—O⁻). Additionally, when sulfur is present in a heteroaryl ring, it may, where the nature of the adjacent atoms and groups permits, exist in an oxidized state (i.e., S⁺—O⁻ or SO₂). Heteroaryl groups may be monocyclic or polycyclic (e.g., bicyclic, tricyclic).

In some instances, a heteroaryl group is monocyclic. Examples include pyrrole, pyrazole, imidazole, triazole (e.g., 1,2,3-triazole, 1,2,4-triazole, 1,2,4-triazole), tetrazole, furan, isoxazole, oxazole, oxadiazole (e.g., 1,2,3-oxadiazole, 1,2,4-oxadiazole, 1,3,4-oxadiazole), thiophene, isothiazole, thiazole, thiadiazole (e.g., 1,2,3-thiadiazole, 1,2,4-thiadiazole, 1,3,4-thiadiazole), pyridine, pyridazine, pyrimidine, pyrazine, triazine (e.g., 1,2,4-triazine, 1,3,5-triazine) and tetrazine.

In some instances, both rings of a polycyclic heteroaryl group are aromatic. Examples include indole, isoindole, indazole, benzoimidazole, benzotriazole, benzofuran, benzoxazole, benzoisoxazole, benzoxadiazole, benzothiophene, benzothiazole, benzoisothiazole, benzothiadiazole, 1H-pyrrolo[2,3-b]pyridine, 1H-pyrazolo[3,4-b]pyridine, 3H-imidazo[4,5-b]pyridine, 3H-[1,2,3]triazolo[4,5-b]pyridine, 1H-pyrrolo[3,2-b]pyridine, 1H-pyrazolo[4,3-b]pyridine, 1H-imidazo[4,5-b]pyridine, 1H-[1,2,3]triazolo[4,5-b]pyridine, 1H-pyrrolo[2,3-c]pyridine, 1H-pyrazolo[3,4-c]pyridine, 3H-imidazo[4,5-c]pyridine, 3H-[1,2,3]triazolo[4,5-c]pyridine, 1H-pyrrolo[3,2-c]pyridine, 1H-pyrazolo[4,3-c]pyridine, 1H-imidazo[4,5-c]pyridine, 1H-[1,2,3]triazolo[4,5-c]pyridine, furo[2,3-b]pyridine, oxazolo[5,4-b]pyridine, isoxazolo[5,4-b]pyridine, [1,2,3]oxadiazolo[5,4-b]pyridine, furo[3,2-b]pyridine, oxazolo[4,5-b]pyridine, isoxazolo[4,5-b]pyridine, [1,2,3]oxadiazolo[4,5-b]pyridine, furo[2,3-c]pyridine, oxazolo[5,4-c]pyridine, isoxazolo[5,4-c]pyridine, [1,2,3]oxadiazolo[5,4-c]pyridine, furo[3,2-c]pyridine, oxazolo[4,5-c]pyridine, isoxazolo[4,5-c]pyridine, [1,2,3]oxadiazolo[4,5-c]pyridine, thieno[2,3-b]pyridine, thiazolo[5,4-b]pyridine, isothiazolo[5,4-b]pyridine, [1,2,3]thiadiazolo[5,4-b]pyridine, thieno[3,2-b]pyridine, thiazolo[4,5-b]pyridine, isothiazolo[4,5-b]pyridine, [1,2,3]thiadiazolo[4,5-b]pyridine, thieno[2,3-c]pyridine, thiazolo[5,4-c]pyridine, isothiazolo[5,4-c]pyridine, [1,2,3]thiadiazolo[5,4-c]pyridine, thieno[3,2-c]pyridine, thiazolo[4,5-c]pyridine, isothiazolo[4,5-c]pyridine, [1,2,3]thiadiazolo[4,5-c]pyridine, quinoline, isoquinoline, cinnoline, quinazoline, quinoxaline, phthalazine, naphthyridine (e.g., 1,8-naphthyridine, 1,7-naphthyridine, 1,6-naphthyridine, 1,5-naphthyridine, 2,7-naphthyridine, 2,6-naphthyridine), imidazo[1,2-a]pyridine, 1H-pyrazolo[3,4-d]thiazole, 1H-pyrazolo[4,3-d]thiazole and imidazo[2,1-b]thiazole.

In other instances, polycyclic heteroaryl groups may include a non-aromatic ring (e.g., cycloalkyl, cycloalkenyl, heterocycloalkyl, heterocycloalkenyl) fused to a heteroaryl ring, provided the polycyclic heteroaryl group is bound to the parent structure via an atom in the aromatic ring. For example, a 4,5,6,7-tetrahydrobenzo[d]thiazol-2-yl group (wherein the moiety is bound to the parent structure via an aromatic carbon atom) is considered a heteroaryl group, while 4,5,6,7-tetrahydrobenzo[d]thiazol-5-yl (wherein the moiety is bound to the parent structure via a non-aromatic carbon atom) is not considered a heteroaryl group.

“Heterocycloalkyl” indicates a non-aromatic, fully saturated ring having, for example, the indicated number of atoms (e.g., 3 to 10, or 3 to 7, membered heterocycloalkyl) made up of one or more heteroatoms (e.g., 1, 2, 3 or 4 heteroatoms) selected from N, O and S and with the remaining ring atoms being carbon. Heterocycloalkyl groups may be monocyclic or polycyclic (e.g., bicyclic, tricyclic).

Examples of monocyclic heterocycloalkyl groups include oxiranyl, aziridinyl, azetidinyl, pyrrolidinyl, imidazolidinyl, pyrazolidinyl, piperidinyl, piperazinyl, morpholinyl and thiomorpholinyl.

When nitrogen is present in a heterocycloalkyl ring, it may, where the nature of the adjacent atoms and groups permits, exist in an oxidized state (i.e., N⁺—O⁻). Examples include piperidinyl N-oxide and morpholinyl-N-oxide. Additionally, when sulfur is present in a heterocycloalkyl ring, it may, where the nature of the adjacent atoms and groups permits, exist in an oxidized state (i.e., S⁺—O⁻ or —SO₂—). Examples include thiomorpholine S-oxide and thiomorpholine S,S-dioxide.

In addition, one ring of a polycyclic heterocycloalkyl group may be aromatic (e.g., aryl or heteroaryl), provided the polycyclic heterocycloalkyl group is bound to the parent structure via a non-aromatic carbon or nitrogen atom. For example, a 1,2,3,4-tetrahydroquinolin-1-yl group (wherein the moiety is bound to the parent structure via a non-aromatic nitrogen atom) is considered a heterocycloalkyl group, while 1,2,3,4-tetrahydroquinolin-8-yl group (wherein the moiety is bound to the parent structure via an aromatic carbon atom) is not considered a heterocycloalkyl group.

“Heterocycloalkenyl” indicates a non-aromatic ring having, for example, the indicated number of atoms (e.g., 3 to 10, or 3 to 7, membered heterocycloalkyl) made up of one or more heteroatoms (e.g., 1, 2, 3 or 4 heteroatoms) selected from N, O and S and with the remaining ring atoms being carbon, and at least one double bond derived by the removal of one molecule of hydrogen from adjacent carbon atoms, adjacent nitrogen atoms, or adjacent carbon and nitrogen atoms of the corresponding heterocycloalkyl. Heterocycloalkenyl groups may be monocyclic or polycyclic (e.g., bicyclic, tricyclic). When nitrogen is present in a heterocycloalkenyl ring, it may, where the nature of the adjacent atoms and groups permits, exist in an oxidized state (i.e., N⁺—O⁻). Additionally, when sulfur is present in a heterocycloalkenyl ring, it may, where the nature of the adjacent atoms and groups permits, exist in an oxidized state (i.e., S⁺—O⁻ or —SO₂—). Examples of heterocycloalkenyl groups include dihydrofuranyl (e.g., 2,3-dihydrofuranyl, 2,5-dihydrofuranyl), dihydrothiophenyl (e.g., 2,3-dihydrothiophenyl, 2,5-dihydrothiophenyl), dihydropyrrolyl (e.g., 2,3-dihydro-1H-pyrrolyl, 2,5-dihydro-1H-pyrrolyl), dihydroimidazolyl (e.g., 2,3-dihydro-1H-imidazolyl, 4,5-dihydro-1H-imidazolyl), pyranyl, dihydropyranyl (e.g., 3,4-dihydro-2H-pyranyl, 3,6-dihydro-2H-pyranyl), tetrahydropyridinyl (e.g., 1,2,3,4-tetrahydropyridinyl, 1,2,3,6-tetrahydropyridinyl) and dihydropyridine (e.g., 1,2-dihydropyridine, 1,4-dihydropyridine). In addition, one ring of a polycyclic heterocycloalkenyl group may be aromatic (e.g., aryl or heteroaryl), provided the polycyclic heterocycloalkenyl group is bound to the parent structure via a non-aromatic carbon or nitrogen atom. For example, a 1,2-dihydroquinolin-1-yl group (wherein the moiety is bound to the parent structure via a non-aromatic nitrogen atom) is considered a heterocycloalkenyl group, while 1,2-dihydroquinolin-8-yl group (wherein the moiety is bound to the parent structure via an aromatic carbon atom) is not considered a heterocycloalkenyl group.

The term “substituted”, as used herein, means that any one or more hydrogens on the designated atom or group is replaced with a selection from the indicated group, provided that the designated atom's normal valence is not exceeded. When a substituent is oxo (i.e., ═O) then 2 hydrogens on the atom are replaced. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation as an agent having at least practical utility. Unless otherwise specified, substituents are named into the core structure. For example, it is to be understood that when (cycloalkyl)alkyl is listed as a possible substituent, the point of attachment of this substituent to the core structure is in the alkyl portion.

The terms “substituted” alkyl (including without limitation C₁-C₄ alkyl), cycloalkyl, aryl, heterocycloalkyl, and heteroaryl, unless otherwise expressly defined, refer respectively to alkyl, cycloalkyl, aryl, heterocycloalkyl, and heteroaryl wherein one or more (such as up to 5, for example, up to 3) hydrogen atoms are replaced by a substituent independently chosen from

—R^{a}, —OR^{b}, —O(C₁-C₂ alkyl)O— (e.g., methylenedioxy-), —SR^{b}, guanidine (—NHC(═NH)NH₂), guanidine wherein one or more of the guanidine hydrogens are replaced with a C₁-C₄ alkyl group, —NR^{b}R^{c}, halo, cyano, oxo (as a substituent for heterocycloalkyl), nitro, —COR^{b}, —CO₂R^{b}, —CONR^{b}R^{c}, —OCOR^{b}, —OCO₂R^{a}, —OCONR^{b}R^{c}, —NR^{c}COR^{b}, —NR^{c}CO₂R^{a}, —NR^{c}CONR^{b}R^{c}, —SOR^{a}, —SO₂R^{a}, —SO₂NR^{b}R^{c}, and —NR^{c}SO₂R^{a},

where R^{a} is chosen from C₁-C₆ alkyl, cycloalkyl, aryl, heterocycloalkyl, and heteroaryl;

R^{b} is chosen from H, C₁-C₆ alkyl, aryl, and heteroaryl; and

R^{c} is chosen from hydrogen and C₁-C₆ alkyl; or

R^{b} and R^{c}, and the nitrogen to which they are attached, form a heterocycloalkyl group; and

where each C₁-C₆ alkyl, cycloalkyl, aryl, heterocycloalkyl, and heteroaryl is optionally substituted with one or more, such as one, two, or three, substituents independently selected from C₁-C₄ alkyl, C₃-C₆ cycloalkyl, aryl, heteroaryl, aryl-C₁-C₄ alkyl-, heteroaryl-C₁-C₄ alkyl-, C₁-C₄ haloalkyl-, —OC₁-C₄ alkyl, —OC₁-C₄ alkylphenyl, —C₁-C₄ alkyl-OH, —C₁-C₄ alkyl-O—C₁-C₄ alkyl, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substituent for heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ phenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

Compounds described herein include, but are not limited to, their optical isomers, racemates, and other mixtures thereof. In those situations, the single enantiomers or diastereomers, i.e., optically active forms, can be obtained by asymmetric synthesis or by resolution of the racemates. Resolution of the racemates can be accomplished, for example, by conventional methods such as crystallization in the presence of a resolving agent, or chromatography, using, for example a chiral high-pressure liquid chromatography (HPLC) column. In addition, such compounds include Z- and E-forms (or cis- and trans-forms) of compounds with carbon-carbon double bonds. Where compounds described herein exist in various tautomeric forms, the term “compound” is intended to include all tautomeric forms of the compound. Such compounds also include crystal forms including polymorphs and clathrates. Similarly, the term “salt” is intended to include all tautomeric forms and crystal forms of the compound.

“Pharmaceutically acceptable salts” include, but are not limited to salts with inorganic acids, such as hydrochloride, phosphate, diphosphate, hydrobromide, sulfate, sulfinate, nitrate, and like salts; as well as salts with an organic acid, such as malate, maleate, fumarate, tartrate, succinate, citrate, acetate, lactate, methanesulfonate, p-toluenesulfonate, 2-hydroxyethylsulfonate, benzoate, salicylate, stearate, and alkanoate such as acetate, HOOC—(CH₂)ₙ—COOH where n is 0-4, and like salts. Similarly, pharmaceutically acceptable cations include, but are not limited to sodium, potassium, calcium, aluminum, lithium, and ammonium.

In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts.

As used herein the terms “group”, “radical” or “fragment” are synonymous and are intended to indicate functional groups or fragments of molecules attachable to a bond or other fragments of molecules.

The term “active agent” is used to indicate a compound or a pharmaceutically acceptable salt thereof which has biological activity. In some embodiments, an “active agent” is a compound or pharmaceutically acceptable salt thereof having pharmaceutical utility. For example an active agent may be an anti-neurodegenerative therapeutic.

The term “therapeutically effective amount” means an amount effective, when administered to a human or non-human patient, to provide a therapeutic benefit such as amelioration of symptoms, slowing of disease progression, or prevention of disease e.g., a therapeutically effective amount may be an amount sufficient to decrease the symptoms of a disease responsive to inhibition of HDAC activity.

As used herein, the terms “histone deacetylase” and “HDAC” are intended to refer to any one of a family of enzymes that remove N^{ε}-acetyl groups from the ε-amino groups of lysine residues of a protein (for example, a histone, or tubulin). Unless otherwise indicated by context, the term “histone” is meant to refer to any histone protein, including H1, H2A, H2B, H3, H4, and H5, from any species. In some embodiments, the histone deacetylase is a human HDAC, including, but not limited to, HDAC-4, HDAC-5, HDAC-6, HDAC-7, HDAC-9, and HDAC-10. In some embodiments, the at least one histone deacetylase is selected from HDAC-4, HDAC-5, HDAC-7, and HDAC-9. In some embodiments, the histone deacetylase is a class Ha HDAC. In some embodiments, the histone deacetylase is HDAC-4. In some embodiments, the histone deacetylase is HDAC-5. In some embodiments, the histone deacetylase is derived from a protozoal or fungal source.

The terms “histone deacetylase inhibitor” and “inhibitor of histone deacetylase” are intended to mean a compound, or a pharmaceutically acceptable salt thereof, described herein which is capable of interacting with a histone deacetylase and inhibiting its enzymatic activity.

The term “a condition or disorder mediated by HDAC” or “a condition or disorder mediated by histone deacetylase” as used herein refers to a condition or disorder in which HDAC and/or the action of HDAC is important or necessary, e.g., for the onset, progress, expression, etc. of that condition, or a condition which is known to be treated by HDAC inhibitors (such as, e.g., trichostatin A).

The term “effect” describes a change or an absence of a change in cell phenotype or cell proliferation. “Effect” can also describe a change or an absence of a change in the catalytic activity of HDAC. “Effect” can also describe a change or an absence of a change in an interaction between HDAC and a natural binding partner.

The term “inhibiting histone deacetylase enzymatic activity” is intended to mean reducing the ability of a histone deacetylase to remove an acetyl group from a protein, such as but not limited to a histone or tubulin. The concentration of inhibitor which reduces the activity of a histone deacetylase to 50% of that of the uninhibited enzyme is determined as the IC₅₀ value. In some embodiments, such reduction of histone deacetylase activity is at least 50%, such as at least about 75%, for example, at least about 90%. In some embodiments, histone deacetylase activity is reduced by at least 95%, such as by at least 99%. In some embodiments, the compounds and pharmaceutical acceptable salts thereof described herein have an IC₅₀ value less than 100 nanomolar. In some embodiments, the compounds and pharmaceutical acceptable salts thereof described herein have an IC₅₀ value from 100 nanomolar to 1 micromolar. In some embodiments, the compounds and pharmaceutical acceptable salts thereof described herein have an IC₅₀ value from 1 to 25 micromolar.

In some embodiments, such inhibition is specific, i.e., the histone deacetylase inhibitor reduces the ability of a histone deacetylase to remove an acetyl group from a protein at a concentration that is lower than the concentration of the inhibitor that is required to produce another, unrelated biological effect. In some embodiments, the concentration of the inhibitor required for histone deacetylase inhibitory activity is at least 2-fold lower, such as at least 5-fold lower, for example, at least 10-fold lower, such as at least 20-fold lower than the concentration required to produce an unrelated biological effect.

“Treatment” or “treating” means any treatment of a disease state in a patient, including

a) preventing the disease, that is, causing the clinical symptoms of the disease not to develop;

b) inhibiting the disease;

c) slowing or arresting the development of clinical symptoms; and/or

d) relieving the disease, that is, causing the regression of clinical symptoms.

“Subject” or “patient” refers to an animal, such as a mammal, that has been or will be the object of treatment, observation or experiment. The methods described herein may be useful in both human therapy and veterinary applications. In some embodiments, the subject is a mammal; and in some embodiments the subject is human.

It is appreciated that certain features described herein, which are, for clarity, described in the context of separate embodiments, may also be provided in combination in a single embodiment. Conversely, various features described herein, which are, for brevity, described in the context of a single embodiment, may also be provided separately or in any suitable subcombination. All combinations of the embodiments pertaining to the chemical groups represented by the variables contained within Formula I, are specifically embraced by herein just as if each and every combination was individually and explicitly recited, to the extent that such combinations embrace compounds that result in stable compounds (i.e., compounds that can be isolated, characterized and tested for biological activity). In addition, all subcombinations of the chemical groups listed in the embodiments describing such variables, as well as all subcombinations of uses and medical indications described herein, such as those conditions or disorders mediated by HDAC, are also specifically embraced herein just as if each and every subcombination of chemical groups and subcombination of uses and medical indications was individually and explicitly recited herein. In addition, some embodiments include every combination of one or more additional agents disclosed herein just as if each and every combination was individually and explicitly recited.

Provided is a compound of Formula I

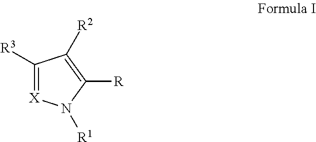

or a pharmaceutically acceptable salt thereof, wherein
X is CR⁴ or N;
R is chosen from —C(O)NH(OH) and —N(OH)C(O)R⁷;
R¹ is optionally substituted aryl or optionally substituted heteroaryl;
R² is chosen from hydrogen, C₁-C₄ alkyl, halo, C₁-C₄ haloalkyl, and nitrile;
R³ is chosen from —OR⁵, —NR⁵R⁶, optionally substituted alkyl, optionally substituted aralkyl, optionally substituted aryl, optionally substituted heteroaryl, optionally substituted heterocycloalkyl, optionally substituted heterocycloalkenyl, optionally substituted cycloalkenyl and optionally substituted cycloalkyl;
R⁴ is chosen from hydrogen, halo, C₁-C₄ alkyl or C₁-C₄ haloalkyl;
R⁵ and R⁶ are independently chosen from hydrogen, optionally substituted C₁-C₄ alkyl, optionally substituted C₁-C₄ haloalkyl, optionally substituted aryl, optionally substituted heteroaryl, optionally substituted heterocycloalkyl, optionally substituted cycloalkyl, optionally substituted aralkyl and optionally substituted heteroaralkyl; or R⁵ and R⁶, together with the nitrogen atom to which they are attached, form an optionally substituted heterocycloalkyl; and
R⁷ is chosen from hydrogen, C₁-C₄ alkyl and C₁-C₄ haloalkyl.

In some embodiments,
R¹ is aryl or heteroaryl, each of which is optionally substituted with 1 to 3 substituents independently chosen from halo, C₁-C₄ alkyl, C₁-C₄ haloalkyl, hydroxyl, alkoxy, and nitrile;
R³ is chosen from —OR⁵, —NR⁵R⁶, alkyl, aralkyl, aryl, heteroaryl, heterocycloalkyl, heterocycloalkenyl, cycloalkenyl and cycloalkyl, wherein the aryl, heteroaryl, heterocycloalkyl, heterocycloalkenyl, cycloalkenyl or cycloalkyl is optionally substituted with 1 to 3 substituents independently chosen from halo, C₁-C₄ alkyl, C₁-C₄ haloalkyl, hydroxyl, alkoxy, nitrile, heteroaryl, phenyl, heterocycloalkyl, cycloalkyl, aralkyl and heteroaralkyl; and
R⁵ and R⁶ are independently chosen from hydrogen, C₁-C₄ alkyl, C₁-C₄ haloalkyl, heteroaryl, heterocycloalkyl, cycloalkyl, aryl, aralkyl and heteroaralkyl, wherein the heteroaryl, heterocycloalkyl, cycloalkyl, aryl, aralkyl or heteroaralkyl is optionally substituted with 1 to 3 substituents independently chosen from halo, C₁-C₄ alkyl, C₁-C₄ haloalkyl, hydroxyl, alkoxy, and nitrile; or R⁵ and R⁶, together with the nitrogen atom to which they are attached, form an optionally substituted heterocycloalkyl comprising one or two heteroatoms.

In some embodiments, R is —C(O)NH(OH).

In some embodiments, R is —N(OH)C(O)R⁷.

In some embodiments, R⁷ is chosen from hydrogen and C₁-C₄ alkyl.

In some embodiments, R⁷ is C₁-C₄ alkyl.

In some embodiments, X is CR⁴.

In some embodiments, R⁴ is hydrogen or C₁-C₄ alkyl.

In some embodiments, R⁴ is hydrogen.

In some embodiments, X is N.

In some embodiments, R¹ is aryl optionally substituted with 1 to 3 substituents independently chosen from halo, C₁-C₄ alkyl, C₁-C₄ haloalkyl, hydroxyl, alkoxy, and nitrile.

In some embodiments, R¹ is phenyl optionally substituted with 1 or 2 substituents independently chosen from C₁-C₄ alkyl and halo.

In some embodiments, R¹ is phenyl.

In some embodiments, R² is chosen from hydrogen, C₁-C₄ alkyl, halo, and C₁-C₄ haloalkyl.

In some embodiments, R² is hydrogen.

In some embodiments, R³ is —OR⁵.

In some embodiments, R⁵ is hydrogen, C₁-C₄ alkyl, or aralkyl.

In some embodiments, R³ is —NR⁵R⁶.

In some embodiments, R⁵ and R⁶, together with the nitrogen atom to which they are attached, form an optionally substituted heterocycloalkyl comprising one or two heteroatoms.

In some embodiments, R⁵ and R⁶, together with the nitrogen atom to which they are attached, form a heterocycloalkyl chosen from pyrrolidin-1-yl, piperazin-1-yl, piperidine-1-yl, and morpholin-4-yl, each of which is optionally substituted with 1 or 2 substituents independently chosen from C₁-C₄ alkyl, C₁-C₄ haloalkyl, cycloalkyl, halo, and phenyl, wherein the phenyl is optionally substituted with 1 or 2 substituents chosen from C₁-C₄ alkyl, C₁-C₄ haloalkyl and halo.

In some embodiments, R⁵ is phenyl optionally substituted with 1 or 2 substituents independently chosen from C₁-C₄ alkyl, C₁-C₄ haloalkyl and halo.

In some embodiments, R⁶ is phenyl optionally substituted with 1 or 2 substituents independently chosen from C₁-C₄ alkyl, C₁-C₄ haloalkyl and halo.

In some embodiments, R³ is optionally substituted aryl.

In some embodiments, R³ is aryl optionally substituted with 1 to 3 substituents independently chosen from halo, C₁-C₄ alkyl, C₁-C₄ haloalkyl, hydroxyl, alkoxy, C₃-C₆ cycloalkyl and nitrile.

In some embodiments, R³ is phenyl, 2,3-dihydrobenzofuran-7-yl, benzo[d][1,3]dioxol-4-yl, chroman-8-yl, 2,3-dihydrobenzo[b][1,4]dioxin-5-yl, and 3,4-dihydro-2H-benzo[b][1,4]oxazin-8-yl, each of which is optionally substituted with 1 to 3 substituents independently chosen from halo, C₁-C₄ alkyl, C₁-C₄ haloalkyl, hydroxyl, alkoxy, C₃-C₆ cycloalkyl and nitrile.

In some embodiments, R³ is optionally substituted heteroaryl.

In some embodiments, R³ is heteroaryl optionally substituted with 1 to 3 substituents independently chosen from halo, C₁-C₄ alkyl, C₁-C₄ haloalkyl, hydroxyl, alkoxy, C₃-C₆ cycloalkyl and nitrile.

In some embodiments, R³ is pyridin-3-yl, benzofuran-7-yl, benzo[b]thiophen-7-yl, and benzo[d]thiazol-4-yl, each of which is optionally substituted with 1 to 3 substituents independently chosen from halo, C₁-C₄ alkyl, C₁-C₄ haloalkyl, hydroxyl, alkoxy, C₃-C₆ cycloalkyl and nitrile.

In some embodiments, R³ is optionally substituted cycloalkyl or optionally substituted cycloalkenyl.

In some embodiments, R³ is cycloalkyl or cycloalkenyl, each of which is optionally substituted with 1 to 3 substituents independently chosen from halo, C₁-C₄ alkyl, C₁-C₄ haloalkyl, hydroxyl, alkoxy, C₃-C₆ cycloalkyl, heteroaryl and nitrile.

In some embodiments, R³ is chosen from cyclopentyl, cyclohexyl, cyclopentenyl and cyclohexenyl, each of which is optionally substituted with 1 to 3 substituents independently chosen from halo, C₁-C₄ alkyl, C₁-C₄ haloalkyl, hydroxyl, alkoxy, C₃-C₆ cycloalkyl, heteroaryl and nitrile.

In some embodiments, R³ is optionally substituted heterocycloalkyl or optionally substituted heterocycloalkenyl.

In some embodiments, R³ is heterocycloalkyl or heterocycloalkenyl, each of which is optionally substituted with 1 to 3 substituents independently chosen from halo, C₁-C₄ alkyl, C₁-C₄ haloalkyl, hydroxyl, alkoxy, C₃-C₆ cycloalkyl, aryl and heteroaryl and nitrile.

In some embodiments, R³ is piperidin-4-yl or 1,2,3,6-tetrahydropyridin-4-yl, each of which is optionally substituted with 1 to 3 substituents independently chosen from halo, C₁-C₄ alkyl, C₁-C₄ haloalkyl, hydroxyl, alkoxy, C₃-C₆ cycloalkyl and nitrile.

In some embodiments, R³ is optionally substituted alkyl.

In some embodiments, R³ is alkyl optionally substituted with 1 to 3 substituents independently chosen from halo, C₁-C₄ alkyl, C₁-C₄ haloalkyl, hydroxyl, alkoxy, C₃-C₆ cycloalkyl, optionally substituted heterocycloalkyl, optionally substituted heteroaryl, and nitrile, wherein the heterocycloalkyl, and heteroaryl groups are optionally substituted with 1 to 3 substituents independently chosen from halo, C₁-C₄ alkyl, C₁-C₄ haloalkyl, hydroxyl, alkoxy, C₃-C₆ cycloalkyl, and nitrile.

In some embodiments, R³ is C₁-C₄ alkyl optionally substituted with 1 to 3 substituents independently chosen from halo, C₁-C₄ alkyl, C₁-C₄ haloalkyl, hydroxyl, alkoxy, C₃-C₆ cycloalkyl, optionally substituted heterocycloalkyl, optionally substituted heteroaryl, and nitrile, wherein the heterocycloalkyl, and heteroaryl groups are optionally substituted with 1 to 3 substituents independently chosen from halo, C₁-C₄ alkyl, C₁-C₄ haloalkyl, hydroxyl, alkoxy, C₃-C₆ cycloalkyl, and nitrile.

In some embodiments, R³ is optionally substituted aralkyl.

In some embodiments, R³ is aralkyl optionally substituted with 1 to 3 substituents independently chosen from halo, C₁-C₄ alkyl, C₁-C₄ haloalkyl, hydroxyl, alkoxy, C₃-C₆ cycloalkyl, optionally substituted heterocycloalkyl, optionally substituted heteroaryl, and nitrile, wherein the heterocycloalkyl, and heteroaryl groups are optionally substituted with 1 to 3 substituents independently chosen from halo, C₁-C₄ alkyl, C₁-C₄ haloalkyl, hydroxyl, alkoxy, C₃-C₆ cycloalkyl, and nitrile.

In some embodiments, R³ is chosen from methylpyridyl, chloropyridyl, phenyl, methylphenyl, chlorophenyl, benzoxy, pyrrolidinyl, cyclopentyl, cyclopentenyl, benzyl, benzothiophenyl, 1-methyl-1,2,3,6-tetrahydropyridin-4-yl, 1-(2,2,2-trifluoroethyl)piperidin-4-yl, 1-isopropylpiperidin-4-yl, 1-cyclopropylpyrrolidin-3-yl, 4-(2,2,2-trifluoroethyl)piperazin-1-yl, 4-isopropylpiperazin-1-yl, 4-cyclopropylpiperazin-1-yl, 4-(2,2,2-trifluoroethyl)piperazin-1-yl)methyl, 1-(4-isopropylpiperazin-1-yl)cyclopropyl, 4-cyclopropylpiperazin-1-yl)methyl, (4-(2,2,2-trifluoroethyl)piperazin-1-yl)methyl, (4-fluorophenyl)(phenyl)methyl, (4-fluorophenyl)(phenyl)amino, 2,3-dihydrobenzofuran-7-yl, 4-methyl-3,4-dihydro-2H-benzo[b][1,4]oxazin-8-yl, and 4-chlorobenzo[d]thiazol-5-yl.

Also provided is a compound chosen from
N-hydroxy-3-(2-methylpyridin-3-yl)-1-phenyl-1H-pyrazole-5-carboxamide,
3-(benzyloxy)-N-hydroxy-1-phenyl-1H-pyrazole-5-carboxamide,
1-(3-fluoro-2-methylphenyl)-N-hydroxy-3-o-tolyl-1H-pyrazole-5-carboxamide,
3-(2-chlorophenyl)-N-hydroxy-1-phenyl-1H-pyrazole-5-carboxamide,
N-hydroxy-1-phenyl-3-(pyrrolidin-1-yl)-1H-pyrazole-5-carboxamide,
3-cyclopentyl-N-hydroxy-1-phenyl-1H-pyrazole-5-carboxamide,
N-hydroxy-1,3-di-o-tolyl-1H-pyrazole-5-carboxamide,
3-cyclopentenyl-N-hydroxy-1-phenyl-1H-pyrazole-5-carboxamide,
3-benzyl-N-hydroxy-1-phenyl-1H-pyrazole-5-carboxamide,
3-(5-chloropyridin-3-yl)-N-hydroxy-1-phenyl-1H-pyrazole-5-carboxamide,
N-hydroxy-1-phenyl-3-o-tolyl-1H-pyrazole-5-carboxamide,
N-hydroxy-1,3-diphenyl-1H-pyrazole-5-carboxamide,
3-(benzo[b]thiophen-7-yl)-N-hydroxy-1-phenyl-1H-pyrazole-5-carboxamide,
N-hydroxy-3-(1-methyl-1,2,3,6-tetrahydropyridin-4-yl)-1-phenyl-1H-pyrazole-5-carboxamide,
4-(2-chlorophenyl)-N-hydroxy-1-phenyl-1H-pyrrole-2-carboxamide,
N-hydroxy-1-phenyl-4-o-tolyl-1H-pyrrole-2-carboxamide;
N-hydroxy-1-phenyl-3-(1-(2,2,2-trifluoroethyl)piperidin-4-yl)-1H-pyrazole-5-carboxamide,
N-hydroxy-3-(1-isopropylpiperidin-4-yl)-1-phenyl-1H-pyrazole-5-carboxamide,
3-(1-cyclopropylpyrrolidin-3-yl)-N-hydroxy-1-phenyl-1H-pyrazole-5-carboxamide,
N-hydroxy-1-phenyl-3-(4-(2,2,2-trifluoroethyl)piperazin-1-yl)-1H-pyrazole-5-carboxamide,
N-hydroxy-3-(4-isopropylpiperazin-1-yl)-1-phenyl-1H-pyrazole-5-carboxamide,
3-(4-cyclopropylpiperazin-1-yl)-N-hydroxy-1-phenyl-1H-pyrazole-5-carboxamide,
N-hydroxy-1-phenyl-3-((4-(2,2,2-trifluoroethyl)piperazin-1-yl)methyl)-1H-pyrazole-5-carboxamide,
N-hydroxy-3-(1-(4-isopropylpiperazin-1-yl)cyclopropyl)-1-phenyl-1H-pyrazole-5-carboxamide,
3-((4-cyclopropylpiperazin-1-yl)methyl)-N-hydroxy-1-phenyl-1H-pyrazole-5-carboxamide,
N-hydroxy-1-phenyl-3-((4-(2,2,2-trifluoroethyl)piperazin-1-yl)methyl)-1H-pyrazole-5-carboxamide,
N-hydroxy-3-(1-(4-isopropylpiperazin-1-yl)cyclopropyl)-1-phenyl-1H-pyrazole-5-carboxamide,
3-((4-cyclopropylpiperazin-1-yl)methyl)-N-hydroxy-1-phenyl-1H-pyrazole-5-carboxamide,
3-((4-fluorophenyl)(phenyl)methyl)-N-hydroxy-1-phenyl-1H-pyrazole-5-carboxamide,
3-((4-fluorophenyl)(phenyl)amino)-N-hydroxy-1-phenyl-1H-pyrazole-5-carboxamide,
3-(2,3-dihydrobenzofuran-7-yl)-N-hydroxy-1-phenyl-1H-pyrazole-5-carboxamide,
N-hydroxy-3-(4-methyl-3,4-dihydro-2H-benzo[b][1,4]oxazin-8-yl)-1-phenyl-1H-pyrazole-5-carboxamide, and
3-(4-chlorobenzo[d]thiazol-5-yl)-N-hydroxy-1-phenyl-1H-pyrazole-5-carboxamide;

or a pharmaceutically acceptable salt thereof.

Methods for obtaining the compounds, or pharmaceutically acceptable salts thereof, described herein will be apparent to those of ordinary skill in the art, suitable procedures being described, for example, in examples below, and in the references cited herein.

Also provided is a method for inhibiting at least one histone deacetylase. Also provided is a use of at least one compound, or pharmaceutically acceptable salt thereof, described herein in the manufacture of medicament for inhibiting at least one histone deacetylase. Also provided is at least one compound, or pharmaceutically acceptable salt thereof, described herein for use in a method for inhibiting at least one histone deacetylase. In some embodiments, at least one histone deacetylase is a class IIa HDAC. In some embodiments, at least one histone deacetylase is selected from HDAC-4, HDAC-5, HDAC-7, and HDAC-9. In some embodiments, the inhibition is in a cell. In some embodiments, the compound, or pharmaceutically acceptable salt thereof, described herein is selective for inhibiting at least one class II histone deacetylase. In some embodiments, the compound, or pharmaceutically acceptable salt thereof, described herein is a selective inhibitor of HDAC-4 and/or HDAC-5.

Also provided is a method of treating a condition or disorder mediated by HDAC in a subject in need of such a treatment, comprising administering to the subject a therapeutically effective amount of at least one compound, or pharmaceutically acceptable salt thereof, described herein. Also provided is a use of at least one compound, or pharmaceutically acceptable salt thereof, described herein in the manufacture of medicament for the treatment of a condition or disorder mediated by HDAC. Also provided is at least one compound, or pharmaceutically acceptable salt thereof, described herein for use in a method for the treatment of the human or animal body by therapy. Also provided is at least one compound, or pharmaceutically acceptable salt thereof, described herein for use in a method for the treatment of a condition or disorder.

In some embodiments, the condition or disorder mediated by HDAC comprises a neurodegenerative pathology. Accordingly, also provided is a method of treating a neurodegenerative pathology mediated by HDAC in a subject in need of such a treatment, comprising administering to the subject a therapeutically effective amount of at least one compound, or pharmaceutically acceptable salt thereof, described herein.

In some embodiments, the neurodegenerative pathology is chosen from Alzheimer's disease, Parkinson's disease, neuronal intranuclear inclusion disease (NIID), Dentatorubral pallidolusyian atrophy (DRPLA), Friedreich's ataxia, Rubenstein-Taubi Sydrome, and polyglutamine diseases such as Huntington's disease; spinocerebellar ataxia 1 (SCA 1), spinocerebellar ataxia 7 (SCA 7), seizures, striatonigral degeneration, progressive supranuclear palsy, torsion dystonia, spasmodic torticollis, dyskinesis, familial tremor, Gilles de la Tourette syndrome, diffuse Lewy body disease, progressive supranuclear palsy, Pick's disease, primary lateral sclerosis, progressive neural muscular atrophy, spinal muscular atrophy, hypertrophic interstitial polyneuropathy, retinitis pigmentosa, hereditary optic atrophy, hereditary spastic paraplegia, Shy-Drager syndrome, Kennedy's disease, protein-aggregation-related neurodegeneration, Machado-Joseph's disease, spongiform encephalopathy, prion-related disease, multiple sclerosis (MS), progressive supranuclear palsy (Steel-Richardson-Olszewski disease), Hallervorden-Spatz disease, progressive familial myoclonic epilepsy, cerebellar degeneration, motor neuron disease, Werdnig-Hoffman disease, Wohlfart-Kugelberg-Welander disease, Charcot-Marie-Tooth disease, Dejerine-Sottas disease, retinitis pigmentosa, Leber's disease, progressive systemic sclerosis, dermatomyositis, and mixed connective tissue disease.

In some embodiments, the neurodegenerative pathology is an acute or chronic degenerative disease of the eye. Acute or chronic degenerative diseases of the eye include glaucoma, dry age-related macular degeneration, retinitis pigmentosa and other forms of heredodegenerative retinal disease, retinal detachment, macular pucker, ischemia affecting the outer retina, cellular damage associated with diabetic retinopathy and retinal ischemia, damage associated with laser therapy, ocular neovascular, diabetic retinopathy, rubeosis iritis, uveitis, Fuch's heterochromatic iridocyclitis, neovascular glaucoma, corneal neovascularization, retinal ischemia, choroidal vascular insufficinency, choroidal thrombosis, carotid artery ischemia, contusive ocular injury, retinopathy of permaturity, retinal vein occlusion, proliferative vitreoretinopathy, corneal angiogenesis, retinal microvasculopathy, and retinal edema.

In some embodiments, the condition or disorder mediated by HDAC comprises a fibrotic disease such as liver fibrosis, cystic fibrosis, cirrhosis, and fibrotic skin diseases, e.g., hypertrophic scars, keloid, and Dupuytren's contracture. Accordingly, also provided is a method of treating a fibrotic disease mediated by HDAC in a subject in need of such a treatment, comprising administering to the subject a therapeutically effective amount of at least one compound, or pharmaceutically acceptable salt thereof, described herein.

In some embodiments, the condition or disorder mediated by HDAC comprises a psychological disorder, such as depression, bipolar disease and dementia. In some embodiments, the condition or disorder mediated by HDAC comprises depression. Accordingly, also provided is a method of treating a psychological disorder, such as depression, mediated by HDAC in a subject in need of such a treatment, comprising administering to the subject a therapeutically effective amount of at least one compound, or pharmaceutically acceptable salt thereof, described herein. In some embodiments, the depression is chosen from major depressive disorder, and bipolar disorder.

In some embodiments, the condition or disorder mediated by HDAC comprises anxiety. Accordingly, also provided is a method of treating an anxiety mediated by HDAC in a subject in need of such a treatment, comprising administering to the subject a therapeutically effective amount of at least one compound, or pharmaceutically acceptable salt thereof, described herein.

In some embodiments, the condition or disorder mediated by HDAC comprises schizophrenia. Accordingly, also provided is a method of treating a schizophrenia mediated by HDAC in a subject in need of such a treatment, comprising administering to the subject a therapeutically effective amount of at least one compound, or pharmaceutically acceptable salt thereof, described herein.

In some embodiments, the condition or disorder mediated by HDAC comprises a motor neuron disease, muscle atrophy/muscle wasting disorders, or amyotrophic lateral sclerosis (ALS). Accordingly, also provided is a method of treating a motor neuron disease, muscle atrophy/muscle wasting disorders, or amyotrophic lateral sclerosis (ALS) mediated by HDAC in a subject in need of such a treatment, comprising administering to the subject a therapeutically effective amount of at least one compound, or pharmaceutically acceptable salt thereof, described herein.

In some embodiments, the condition or disorder mediated by HDAC comprises a cardiovascular condition. Accordingly, also provided is a method of treating a cardiovascular condition mediated by HDAC in a subject in need of such a treatment, comprising administering to the subject a therapeutically effective amount of at least one compound, or pharmaceutically acceptable salt thereof, described herein. In some embodiments, the cardiovascular condition is chosen from cardiomyopathy, cardiac hypertrophy, myocardial ischemia, heart failure, cardiac restenosis, and arteriosclerosis.

In some embodiments, the condition or disorder mediated by HDAC comprises cancer. Accordingly, also provided is a method of treating cancer mediated by HDAC in a subject in need of such a treatment, comprising administering to the subject a therapeutically effective amount of at least one compound, or pharmaceutically acceptable salt thereof, described herein. In some embodiments, the cancer is chosen from lymphoma, pancreatic cancer, colorectal cancer, hepatocellular carcinoma, Waldenstrom macroglobulinemia, hormone refractory cancer of the prostate, and leukaemia, breast cancer, lung cancer, ovarian cancer, prostate cancer, head and neck cancer, renal cancer, gastric cancer, brain cancer, B-cell lymphoma, peripheral T-cell lymphoma, and cutaneous T-cell lymphoma. In some further embodiments, the cancer is chosen from the following cancer types. Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma, cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma; and the sensitization of tumors to radiotherapy by administering the compound according to the invention before, during or after irradiation of the tumor for treating cancer.

In some embodiments, the condition or disorder mediated by HDAC comprises a condition or disorder treatable by immune modulation. Accordingly, also provided is a method of treating a condition or disorder treatable by immune modulation mediated by HDAC in a subject in need of such a treatment, comprising administering to the subject a therapeutically effective amount of at least one compound, or pharmaceutically acceptable salt thereof, described herein. In some embodiments, the condition or disorder treatable by immune modulation is chosen from asthma, irritable bowel syndrome, Crohn's disease, ulcerative colitis, bowel motility disorders, hypertension, rheumatoid arthritis, osteoarthritis, juvenile chronic arthritis, graft versus host disease, psoriasis, spondyloarthropathy, inflammatory bowel disease, alcoholic hepatitis, Sjogren's syndrome, ankylosing spondylitis, membranous glomerulopathy, discogenic pain, systemic lupus erythematosus, allergic bowel disease, coeliac disease, bronchitis, cystic fibrosis, rheumatoid spondylitis, osteoarthritis, uveitis, iritis, and conjunctivitis, ischemic bowel disease, psoriasis, eczema, dermatitis, septic arthritis, gout, pseudogout, juvenile arthritis, Still's disease, Henoch-Schonlein purpura, psoriatic arthritis, myalgia, reactive arthritis (Reiter's syndrome), hemochromatosis, Wegener's granulomatosis, familial Mediterranean fever (FMF), HBDS (hyperimmunoglobulinemia D and periodic fever syndrome), TRAPS (TNF-alpha receptor associated periodic fever syndrome), chronic obstructive pulmonary disease, neonatal-onset multisystem inflammatory disease (NOMID), cryopyrin-associated periodic syndrome (CAPS), and familial cold autoinflammatory syndrome (FCAS).

In some embodiments, the condition or disorder mediated by HDAC comprises an allergic disease. Accordingly, also provided is a method of treating an allergic disease, mediated by HDAC in a subject in need of such a treatment, comprising administering to the subject a therapeutically effective amount of at least one compound, or pharmaceutically acceptable salt thereof, described herein. Allergic diseases include, but are not limited to, respiratory allergic diseases such as allergic rhinitis, hypersensitivity lung diseases, hypersensitivity pneumonitis, eosinophilic pneumonias, Loeffler's syndrome, chronic eosinophilic pneumonia, delayed-type hypersensitivity, interstitial lung diseases (ILD), idiopathic pulmonary fibrosis, polymyositis, dermatomyositis, systemic anaphylaxis, drug allergies (e.g., to penicillin or cephalosporins), and insect sting allergies.

In some embodiments, the condition or disorder mediated by HDAC comprises an infectious disease such as a fungal infection, bacterial infection, viral infection, and protozoal infection, e.g., malaria, giardiasis, leishmaniasis, Chaga's disease, dysentery, toxoplasmosis, and coccidiosis. In some embodiments, the condition or disorder mediated by HDAC comprises malaria. Accordingly, also provided is a method of treating an infectious disease, such as malaria, mediated by HDAC in a subject in need of such a treatment, comprising administering to the subject a therapeutically effective amount of at least one compound, or pharmaceutically acceptable salt thereof, described herein.

In some embodiments, the condition or disorder mediated by HDAC comprises autism or Rett syndrome. Accordingly, also provided is a method of treating autism or Rett syndrome mediated by HDAC in a subject in need of such a treatment, comprising administering to the subject a therapeutically effective amount of at least one compound, or pharmaceutically acceptable salt thereof, described herein.

In some embodiments, the condition or disorder mediated by HDAC comprises a hematological disorder such as thalassemia, anemia, and sickle cell anemia. Accordingly, also provided is a method of treating a hematological disorder mediated by HDAC in a subject in need of such a treatment, comprising administering to the subject a therapeutically effective amount of at least one compound, or pharmaceutically acceptable salt thereof, described herein.

In some embodiments, the condition or disorder mediated by HDAC comprises a metabolic disease such as prediabetes or diabetes (type I or II). Accordingly, also provided is a method of treating a metabolic disease, such as prediabetes or diabetes (type I or II), mediated by HDAC in a subject in need of such a treatment, comprising administering to the subject a therapeutically effective amount of at least one compound, or pharmaceutically acceptable salt thereof, described herein.

In some embodiments, the condition or disorder mediated by HDAC comprises a disorder that may also be treated by progenitor/stem cell based therapies such as: disorders related to diabetes (organ failure, cirrhosis, and hepatitis); central nervous system (CNS) disorders associated with dysregulation of progenitor cells in the brain (e.g., post-traumatic stress disorder (PTSD); tumors (e.g., retinoblastomas); disorders affecting oligodendrocyte progenitor cells (e.g., astrocytomas and ependimal cell tumors); multiple sclerosis; demyelinating disorders such as the leukodystrophies; neuropathies associated with white matter loss; and cerebellar disorders such as ataxia; and olfactory progenitor disorders (e.g., anosmic conditions). Accordingly, also provided is a method of treating a disorder that is mediated by HDAC in a subject in need of such a treatment, comprising administering to the subject a therapeutically effective amount of at least one compound, or pharmaceutically acceptable salt thereof, described herein, either before, during, or after a treatment with progenitor/stem cell based therapies.

In some embodiments, the condition or disorder mediated by HDAC comprises a disorder related to the proliferation of epithelial and mesenchymal cells (e.g., tumors, wound healing, and surgeries). Accordingly, also provided is a method of treating a disorder related to the proliferation of epithelial and mesenchymal cells that is mediated by HDAC in a subject in need of such a treatment, comprising administering to the subject a therapeutically effective amount of at least one compound, or pharmaceutically acceptable salt thereof, described herein.

In some embodiments, the condition or disorder mediated by HDAC comprises a disorder related to the proliferation of bone progenitors (e.g., osteoblasts and osteoclasts), disorders related to hair and epidermal progenitors (e.g., hair loss, cutaneous tumors, skin regeneration, burns, and cosmetic surgery); and disorders related to bone loss during menopause. Accordingly, also provided is a method of treating disorders related to the proliferation of bone progenitors, disorders related to hair and epidermal progenitors, or disorders related to bone loss that are mediated by HDAC in a subject in need of such a treatment, comprising administering to the subject a therapeutically effective amount of at least one compound, or pharmaceutically acceptable salt thereof, described herein.

In some embodiments, the condition or disorder mediated by HDAC is a viral disorder for which blood cells become sensitized to other treatments after HDAC inhibition, following administering to the subject a therapeutically effective amount of at least one compound, or pharmaceutically acceptable salt thereof, as described herein. Accordingly, also provided is a method of treating a viral disorder, wherein blood cells become sensitized to other treatments after HDAC inhibition, that is mediated by HDAC in a subject in need of such a treatment, comprising administering to the subject a therapeutically effective amount of at least one compound, or pharmaceutically acceptable salt thereof, described herein.

In some embodiments, the condition or disorder mediated by HDAC is an immune disorder that may be co-treated with TNFα or other immune modulators, upon administering to the subject a therapeutically effective amount of at least one compound, or pharmaceutically acceptable salt thereof, as described herein. Accordingly, also provided is a method of treating an immune disorder that is mediated by HDAC in a subject in need of such a treatment, comprising administering to the subject a therapeutically effective amount of at least one compound, or pharmaceutically acceptable salt thereof, described herein, either before, during, or after a treatment with TNFα or other immune modulators.

In some embodiments, the condition or disorder mediated by HDAC comprises a graft rejection or transplant rejection. Accordingly, also provided is a method of treating a disorder related to a graft rejection or a transplant rejection that is mediated by HDAC in a subject in need of such a treatment, comprising administering to the subject a therapeutically effective amount of at least one compound, or pharmaceutically acceptable salt thereof, described herein.

In some embodiments, the condition or disorder mediated by HDAC comprises a blood pressure disorder related to nitric oxide (NO) regulation (e.g., hypertension, erectile dysfunction, asthma; and ocular disorders as glaucoma). Accordingly, also provided is a method of treating a blood pressure disorder related to nitric oxide (NO) regulation that is mediated by HDAC in a subject in need of such a treatment, comprising administering to the subject a therapeutically effective amount of at least one compound, or pharmaceutically acceptable salt thereof, described herein. In some embodiments, the condition or disorder is a cardiac hypertrophic disorder. Accordingly, also provided is a method of treating a cardiac hypertrophic disorder that is mediated by HDAC in a subject in need of such a treatment, comprising administering to the subject a therapeutically effective amount of at least one compound, or pharmaceutically acceptable salt thereof, described herein.

Also provided are methods of treatment in which at least one compound, or pharmaceutically acceptable salt thereof, described herein is the only active agent given to the subject and methods of treatment in which at least one compound, or pharmaceutically acceptable salt thereof, described herein is given to the subject in combination with one or more additional active agents.

In general, the compounds, or pharmaceutically acceptable salts thereof, described herein will be administered in a therapeutically effective amount by any of the accepted modes of administration for agents that serve similar utilities. The actual amount of the compound, i.e., the active ingredient, will depend upon numerous factors such as the severity of the disease to be treated, the age and relative health of the subject, the potency of the compound used, the route and form of administration, and other factors well known to the skilled artisan. The drug can be administered at least once a day, such as once or twice a day.

In some embodiments, the compounds, or pharmaceutically acceptable salts thereof, described herein are administered as a pharmaceutical composition. Accordingly, provided are pharmaceutical compositions comprising at least one compound, or pharmaceutically acceptable salt thereof, described herein, together with at least one pharmaceutically acceptable vehicle chosen from carriers, adjuvants, and excipients. A compound of the present invention can be formulated into pharmaceutical compositions using techniques well known to those in the art.

Pharmaceutically acceptable vehicles must be of sufficiently high purity and sufficiently low toxicity to render them suitable for administration to the animal being treated. The vehicle can be inert or it can possess pharmaceutical benefits. The amount of vehicle employed in conjunction with the compound, or pharmaceutically acceptable salt thereof, is sufficient to provide a practical quantity of material for administration per unit dose of the compound, or pharmaceutically acceptable salt thereof.

Exemplary pharmaceutically acceptable carriers or components thereof are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; synthetic oils; vegetable oils, such as peanut oil, cottonseed oil, sesame oil, olive oil, and corn oil; polyols such as propylene glycol, glycerine, sorbitol, mannitol, and polyethylene glycol; alginic acid; phosphate buffer solutions; emulsifiers, such as the TWEENs®; wetting agents, such sodium lauryl sulfate; coloring agents; flavoring agents; tableting agents; stabilizers; antioxidants; preservatives; pyrogen-free water; isotonic saline; and phosphate buffer solutions.

Optional active agents may be included in a pharmaceutical composition, which do not substantially interfere with the activity of the compound, or pharmaceutically acceptable salt thereof, described herein.

Effective concentrations of at least one compound, or pharmaceutically acceptable salt thereof, described herein are mixed with a suitable pharmaceutically acceptable vehicle. In instances in which the compound, or pharmaceutically acceptable salt thereof, exhibits insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate.

Upon mixing or addition of a compound, or pharmaceutically acceptable salt thereof, described herein, the resulting mixture may be a solution, suspension, emulsion or the like. The form of the resulting mixture depends upon a number of factors, including the intended mode of administration and the solubility of the compound, or pharmaceutically acceptable salt thereof, in the chosen vehicle. The effective concentration sufficient for ameliorating the symptoms of the disease treated may be empirically determined.

The compounds, or pharmaceutically acceptable salts thereof, described herein may be administered orally, topically, parenterally, intravenously, by intramuscular injection, by inhalation or spray, sublingually, transdermally, via buccal administration, rectally, as an ophthalmic solution, or by other means, in dosage unit formulations.

Pharmaceutical compositions may be formulated for oral use, such as for example, tablets, troches, lozenges, aqueous or oily suspensions, dispersible powders or granules, emulsions, hard or soft capsules, or syrups or elixirs. Pharmaceutical compositions intended for oral use may be prepared according to any method known to the art for the manufacture of pharmaceutical compositions and such compositions may contain one or more agents, such as sweetening agents, flavoring agents, coloring agents and preserving agents, in order to provide pharmaceutically elegant and palatable preparations. In some embodiments, oral pharmaceutical compositions contain from 0.1 to 99% of at least one compound, or pharmaceutically acceptable salt thereof, described herein. In some embodiments, oral pharmaceutical compositions contain at least 5% (weight %) of at least one compound, or pharmaceutically acceptable salt thereof, described herein. Some embodiments contain from 25% to 50% or from 5% to 75% of at least one compound, or pharmaceutically acceptable salt thereof, described herein.

Orally administered pharmaceutical compositions also include liquid solutions, emulsions, suspensions, powders, granules, elixirs, tinctures, syrups, and the like. The pharmaceutically acceptable carriers suitable for preparation of such compositions are well known in the art. Oral pharmaceutical compositions may contain preservatives, flavoring agents, sweetening agents, such as sucrose or saccharin, taste-masking agents, and coloring agents.

Typical components of carriers for syrups, elixirs, emulsions and suspensions include ethanol, glycerol, propylene glycol, polyethylene glycol, liquid sucrose, sorbitol and water. Syrups and elixirs may be formulated with sweetening agents, for example glycerol, propylene glycol, sorbitol or sucrose. Such pharmaceutical compositions may also contain a demulcent.

The compound, or pharmaceutically acceptable salt thereof, described herein can be incorporated into oral liquid preparations such as aqueous or oily suspensions, solutions, emulsions, syrups, or elixirs, for example. Moreover, pharmaceutical compositions containing the compound, or pharmaceutically acceptable salt thereof, described herein can be presented as a dry product for constitution with water or other suitable vehicle before use. Such liquid preparations can contain conventional additives, such as suspending agents (e.g., sorbitol syrup, methyl cellulose, glucose/sugar, syrup, gelatin, hydroxyethyl cellulose, carboxymethyl cellulose, aluminum stearate gel, and hydrogenated edible fats), emulsifying agents (e.g., lecithin, sorbitan monooleate, or acacia), non-aqueous vehicles, which can include edible oils (e.g., almond oil, fractionated coconut oil, say′ esters, propylene glycol and ethyl alcohol), and preservatives (e.g., methyl or propyl p-hydroxybenzoate and sorbic acid).

For a suspension, typical suspending agents include methylcellulose, sodium carboxymethyl cellulose, AVICEL® RC-591, tragacanth and sodium alginate; typical wetting agents include lecithin and polysorbate 80; and typical preservatives include methyl paraben and sodium benzoate.

Aqueous suspensions contain the active material(s) in admixture with excipients suitable for the manufacture of aqueous suspensions. Such excipients are suspending agents, for example sodium carboxymethylcellulose, methylcellulose, hydropropylmethylcellulose, sodium alginate, polyvinylpyrrolidone, gum tragacanth and gum acacia; dispersing or wetting agents; may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol substitute, or condensation products of ethylene oxide with partial esters derived from fatty acids and hexitol anhydrides, for example polyethylene sorbitan substitute. The aqueous suspensions may also contain one or more preservatives, for example ethyl, or n-propyl p-hydroxybenzoate.

Oily suspensions may be formulated by suspending the active ingredients in a vegetable oil, for example peanut oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These pharmaceutical compositions may be preserved by the addition of an anti-oxidant such as ascorbic acid.

Pharmaceutical compositions may also be in the form of oil-in-water emulsions. The oily phase may be a vegetable oil, for example olive oil or peanut oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above.

Tablets typically comprise conventional pharmaceutically acceptable adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, can be useful adjuvants for chewable tablets. Capsules (including time release and sustained release formulations) typically comprise one or more solid diluents disclosed above. The selection of carrier components often depends on secondary considerations like taste, cost, and shelf stability.

Such pharmaceutical compositions may also be coated by conventional methods, typically with pH or time-dependent coatings, such that the compound, or pharmaceutically acceptable salt thereof, is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methylcellulose phthalate, ethyl cellulose, Eudragit® coatings, waxes and shellac.

Pharmaceutical compositions for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be sterile injectable solution or suspension in a non-toxic parentally acceptable vehicle, for example as a solution in 1,3-butanediol. Among the acceptable vehicles that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid can be useful in the preparation of injectables.

The compound, or pharmaceutically acceptable salt thereof, described herein may be administered parenterally in a sterile medium. Parenteral administration includes subcutaneous injections, intravenous, intramuscular, intrathecal injection or infusion techniques. The compound, or pharmaceutically acceptable salt thereof, described herein, depending on the vehicle and concentration used, can either be suspended or dissolved in the vehicle. Advantageously, adjuvants such as local anesthetics, preservatives and buffering agents can be dissolved in the vehicle. In many pharmaceutical compositions for parenteral administration the carrier comprises at least 90% by weight of the total composition. In some embodiments, the carrier for parenteral administration is chosen from propylene glycol, ethyl oleate, pyrrolidone, ethanol, and sesame oil.

The compound, or pharmaceutically acceptable salt thereof, described herein may also be administered in the form of suppositories for rectal administration of the drug. These pharmaceutical compositions can be prepared by mixing the drug with a suitable non-irritating excipient that is solid at ordinary temperatures but liquid at rectal temperature and will therefore melt in the rectum to release the drug. Such materials include cocoa butter and polyethylene glycols.

The compound, or pharmaceutically acceptable salt thereof, described herein may be formulated for local or topical application, such as for topical application to the skin and mucous membranes, such as in the eye, in the form of gels, creams, and lotions and for application to the eye. Topical pharmaceutical compositions may be in any form including, for example, solutions, creams, ointments, gels, lotions, milks, cleansers, moisturizers, sprays, skin patches, and the like.

Such solutions may be formulated as 0.01%-10% isotonic solutions, pH 5-7, with appropriate salts. The compound, or pharmaceutically acceptable salt thereof, described herein may also be formulated for transdermal administration as a transdermal patch.

Topical pharmaceutical compositions comprising at least one compound, or pharmaceutically acceptable salt thereof, described herein can be admixed with a variety of carrier materials well known in the art, such as, for example, water, alcohols, aloe vera gel, allantoin, glycerine, vitamin A and E oils, mineral oil, propylene glycol, PPG-2 myristyl propionate, and the like.

Other materials suitable for use in topical carriers include, for example, emollients, solvents, humectants, thickeners and powders. Examples of each of these types of materials, which can be used singly or as mixtures of one or more materials, are as follows.

Representative emollients include stearyl alcohol, glyceryl monoricinoleate, glyceryl monostearate, propane-1,2-diol, butane-1,3-diol, mink oil, cetyl alcohol, iso-propyl isostearate, stearic acid, iso-butyl palmitate, isocetyl stearate, oleyl alcohol, isopropyl laurate, hexyl laurate, decyl oleate, octadecan-2-ol, isocetyl alcohol, cetyl palmitate, dimethylpolysiloxane, di-n-butyl sebacate, iso-propyl myristate, iso-propyl palmitate, iso-propyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, and myristyl myristate; propellants, such as propane, butane, iso-butane, dimethyl ether, carbon dioxide, and nitrous oxide; solvents, such as ethyl alcohol, methylene chloride, iso-propanol, castor oil, ethylene glycol monoethyl ether, diethylene glycol monobutyl ether, diethylene glycol monoethyl ether, dimethyl sulphoxide, dimethyl formamide, tetrahydrofuran; humectants, such as glycerin, sorbitol, sodium 2-pyrrolidone-5-carboxylate, soluble collagen, dibutyl phthalate, and gelatin; and powders, such as chalk, talc, fullers earth, kaolin, starch, gums, colloidal silicon dioxide, sodium polyacrylate, tetra alkyl ammonium smectites, trialkyl aryl ammonium smectites, chemically modified magnesium aluminium silicate, organically modified montmorillonite clay, hydrated aluminium silicate, fumed silica, carboxyvinyl polymer, sodium carboxymethyl cellulose, and ethylene glycol monostearate.

The compound, or pharmaceutically acceptable salt thereof, described herein may also be topically administered in the form of liposome delivery systems, such as small unilamellar vesicles, large unilamellar vesicles, and multilamellar vesicles. Liposomes can be formed from a variety of phospholipids, such as cholesterol, stearylamine or phosphatidylcholines.

Other pharmaceutical compositions useful for attaining systemic delivery of the compound, or pharmaceutically acceptable salt thereof, include sublingual, buccal and nasal dosage forms. Such pharmaceutical compositions typically comprise one or more of soluble filler substances such as sucrose, sorbitol and mannitol, and binders such as acacia, microcrystalline cellulose, carboxymethyl cellulose, and hydroxypropyl methylcellulose. Glidants, lubricants, sweeteners, colorants, antioxidants and flavoring agents disclosed above may also be included.

Pharmaceutical compositions for inhalation typically can be provided in the form of a solution, suspension or emulsion that can be administered as a dry powder or in the form of an aerosol using a conventional propellant (e.g., dichlorodifluoromethane or trichlorofluoromethane).

The pharmaceutical compositions may also optionally comprise an activity enhancer. The activity enhancer can be chosen from a wide variety of molecules that function in different ways to enhance or be independent of therapeutic effects of the compound, or pharmaceutically acceptable salt thereof, described herein. Particular classes of activity enhancers include skin penetration enhancers and absorption enhancers.

Pharmaceutical compositions may also contain additional active agents that can be chosen from a wide variety of molecules, which can function in different ways to enhance the therapeutic effects of at least one compound, or pharmaceutically acceptable salt thereof, described herein. These optional other active agents, when present, are typically employed in the pharmaceutical compositions at a level ranging from 0.01% to 15%. Some embodiments contain from 0.1% to 10% by weight of the composition. Other embodiments contain from 0.5% to 5% by weight of the composition.

Also provided are packaged pharmaceutical compositions. Such packaged compositions include a pharmaceutical composition comprising at least one compound, or pharmaceutically acceptable salt thereof, described herein, and instructions for using the composition to treat a subject (typically a human patient). In some embodiments, the instructions are for using the pharmaceutical composition to treat a subject suffering a condition or disorder mediated by HDAC. The packaged pharmaceutical composition can include providing prescribing information; for example, to a patient or health care provider, or as a label in a packaged pharmaceutical composition. Prescribing information may include for example efficacy, dosage and administration, contraindication and adverse reaction information pertaining to the pharmaceutical composition.

In all of the foregoing the compound, or pharmaceutically acceptable salt thereof, can be administered alone, as mixtures, or in combination with other active agents.

The methods described herein include methods for treating Huntington's disease, including treating memory and/or cognitive impairment associated with Huntington's disease, comprising administering to a subject, simultaneously or sequentially, at least one compound, or pharmaceutically acceptable salt thereof, described herein and one or more additional agents used in the treatment of Huntington's disease such as, but not limited to, Amitriptyline, Imipramine, Desipramine, Nortriptyline, Paroxetine, Fluoxetine, Sertraline, Tetrabenazine, Haloperidol, Chlorpromazine, Thioridazine, Sulpride, Quetiapine, Clozapine, and Risperidone. In methods using simultaneous administration, the agents can be present in a combined composition or can be administered separately. As a result, also provided are pharmaceutical compositions comprising at least one compound, or pharmaceutically acceptable salt thereof, described herein and one or more additional pharmaceutical agents used in the treatment of Huntington's disease such as, but not limited to, Amitriptyline, Imipramine, Desipramine, Nortriptyline, Paroxetine, Fluoxetine, Sertraline, Tetrabenazine, Haloperidol, Chlorpromazine, Thioridazine, Sulpride, Quetiapine, Clozapine, and Risperidone. Similarly, also provided are packaged pharmaceutical compositions containing a pharmaceutical composition comprising at least one compound, or pharmaceutically acceptable salt thereof, described herein, and another composition comprising one or more additional pharmaceutical agents used in the treatment of Huntington's disease such as, but not limited to, Amitriptyline, Imipramine, Desipramine, Nortriptyline, Paroxetine, Fluoxetine, Sertraline, Tetrabenazine, Haloperidol, Chlorpromazine, Thioridazine, Sulpride, Quetiapine, Clozapine, and Risperidone.

Also provided are methods for treating Alzheimer's disease, including treating memory and/or cognitive impairment associated with Alzheimer's disease, comprising administering to a subject, simultaneously or sequentially, at least one compound, or pharmaceutically acceptable salt thereof, described herein and one or more additional agents used in the treatment of Alzheimer's disease such as, but not limited to, Reminyl®, Cognex®, Aricept®, Exelon®, Akatinol®, Neotropin, Eldepryl®, Estrogen and Clioquinol. In methods using simultaneous administration, the agents can be present in a combined composition or can be administered separately. Also provided are pharmaceutical compositions comprising at least one compound, or pharmaceutically acceptable salt thereof, described herein, and one or more additional pharmaceutical agents used in the treatment of Alzheimer's disease such as, but not limited to, Reminyl®, Cognex®, Aricept®, Exelon®, Akatinol®, Neotropin™, Eldepryl®, Estrogen and Clioquinol. Similarly, also provided are packaged pharmaceutical compositions containing a pharmaceutical composition comprising at least one compound, or pharmaceutically acceptable salt thereof, described herein, and another composition comprising one or more additional pharmaceutical agents used in the treatment of Alzheimer's disease such as, but not limited to Reminyl®, Cognex®, Aricept®, Exelon®, Akatinol®, Neotropin™, Eldepryl®, Estrogen and Clioquinol.

Also provided are methods for treating cancer comprising administering to a subject, simultaneously or sequentially, at least one compound, or pharmaceutically acceptable salt thereof, described herein and one or more additional agents used in the treatment of cancer such as, but not limited to, the following categories of anti-tumor agents:

(i) other cell cycle inhibitory agents that work by the same or different mechanisms from those defined hereinbefore, for example cyclin dependent kinase (CDK) inhibitors, in particular CDK2 inhibitors;

(ii) cytostatic agents such as antioestrogens (for example tamoxifen, toremifene, raloxifene, droloxifene, iodoxyfene), progestogens (for example megestrol acetate), aromatase inhibitors (for example anastrozole, letrazole, vorazole, exemestane), antiprogestogens, antiandrogens (for example flutamide, nilutamide, bicalutamide, cyproterone acetate), LHRH agonists and antagonists (for example goserelin acetate, luprolide), inhibitors of testosterone 5α-dihydroreductase (for example finasteride), anti-invasion agents (for example metalloproteinase inhibitors like marimastat and inhibitors of urokinase plasminogen activator receptor function) and inhibitors of growth factor function, (such growth factors include for example vascular endothelial growth factor, epithelial growth factor, platelet derived growth factor and hepatocyte growth factor such inhibitors include growth factor antibodies, growth factor receptor antibodies, tyrosine kinase inhibitors and serine/threonine kinase inhibitors);

(iii) antiproliferative/antineoplastic drugs and combinations thereof, as used in medical oncology, such as antimetabolites (for example antifolates like methotrexate, fluoropyrimidines like 5-fluorouracil, purine and adenosine analogues, cytosine arabinoside); antitumour antibiotics (for example anthracyclines like doxorubicin, daunomycin, epirubicin and idarubicin, mitomycin-C, dactinomycin, mithramycin); platinum derivatives (for example cisplatin, carboplatin); alkylating agents (for example nitrogen mustard, melphalan, chlorambucil, busulphan, cyclophosphamide, ifosfamide, nitrosoureas, thiotepa); antimitotic agents (for example vinca alkaloids like vincrisitine and taxoids like taxol, taxotere); topoisomerase inhibitors (for example epipodophyllotoxins like etoposide and teniposide, amsacrine, topotecan);

(iv) antiangiogenic agents that work by different mechanisms from those defined hereinbefore (for example receptor tyrosine kinases like Tie-2, inhibitors of integrin α_{V}β₃ function, angiostatin, razoxin, thalidomide), and including vascular targeting agents; and

(v) differentiation agents (for example retinoic acid and vitamin D).

In methods using simultaneous administration, the agents can be present in a combined composition or can be administered separately. Also provided are pharmaceutical compositions comprising at least one compound, or pharmaceutically acceptable salt thereof, described herein, and one or more anti-tumor agent as described herein. Similarly, also provided are packaged pharmaceutical compositions containing a pharmaceutical composition comprising at least one compound, or pharmaceutically acceptable salt thereof, described herein, and another composition comprising one or more one or more anti-tumor agent as described herein. When used in combination with one or more additional pharmaceutical agent or agents, the described herein may be administered prior to, concurrently with, or following administration of the additional pharmaceutical agent or agents.

In some embodiments, the compounds, or pharmaceutically acceptable salts thereof, described herein, are administered in conjunction with surgery or radiotherapy, optionally in combination with one or more additional agents used in the treatment of cancer.

The dosages of the compounds described herein depend upon a variety of factors including the particular syndrome to be treated, the severity of the symptoms, the route of administration, the frequency of the dosage interval, the particular compound utilized, the efficacy, toxicology profile, pharmacokinetic profile of the compound, and the presence of any deleterious side-effects, among other considerations.

The compound, or pharmaceutically acceptable salt thereof, described herein is typically administered at dosage levels and in a manner customary for HDAC inhibitors. For example, the compound, or pharmaceutically acceptable salt thereof, can be administered, in single or multiple doses, by oral administration at a dosage level of generally 0.001-100 mg/kg/day, for example, 0.01-100 mg/kg/day, such as 0.1-70 mg/kg/day, for example, 0.5-10 mg/kg/day. Unit dosage forms can contain generally 0.01-1000 mg of at least one compound, or pharmaceutically acceptable salt thereof, described herein, for example, 0.1-50 mg of at least one compound, or pharmaceutically acceptable salt thereof, described herein. For intravenous administration, the compounds can be administered, in single or multiple dosages, at a dosage level of, for example, 0.001-50 mg/kg/day, such as 0.001-10 mg/kg/day, for example, 0.01-1 mg/kg/day. Unit dosage forms can contain, for example, 0.1-10 mg of at least one compound, or pharmaceutically acceptable salt thereof, described herein.

A labeled form of a compound, or pharmaceutically acceptable salt thereof, described herein can be used as a diagnostic for identifying and/or obtaining compounds that have the function of modulating an activity of HDAC as described herein. The compound, or pharmaceutically acceptable salt thereof, described herein may additionally be used for validating, optimizing, and standardizing bioassays.

By “labeled” herein is meant that the compound is either directly or indirectly labeled with a label which provides a detectable signal, e.g., radioisotope, fluorescent tag, enzyme, antibodies, particles such as magnetic particles, chemiluminescent tag, or specific binding molecules, etc. Specific binding molecules include pairs, such as biotin and streptavidin, digoxin and antidigoxin etc. For the specific binding members, the complementary member would normally be labeled with a molecule which provides for detection, in accordance with known procedures, as outlined above. The label can directly or indirectly provide a detectable signal.

The present disclosure includes all isotopes of atoms occurring in the compounds and pharmaceutically acceptable salts thereof described herein. Isotopes include those atoms having the same atomic number but different mass numbers. The present disclosure also includes every combination of one or more atoms in the compounds and pharmaceutically acceptable salts thereof described herein that is replaced with an atom having the same atomic number but a different mass number. One such example is the replacement of an atom that is the most naturally abundant isotope, such as ¹H or ¹²C, found in one of the compounds and pharmaceutically acceptable salts thereof described herein, with a different atom that is not the most naturally abundant isotope, such as ²H or ³H (replacing ¹H), or ¹¹C, ¹³C, or ¹²C (replacing ¹²C). A compound wherein such a replacement has taken place is commonly referred to as being isotopically-labeled. Isotopic-labeling of the compounds and pharmaceutically acceptable salts thereof described herein can be accomplished using any one of a variety of different synthetic methods know to those of ordinary skill in the art and they are readily credited with understanding the synthetic methods and available reagents needed to conduct such isotopic-labeling. By way of general example, and without limitation, isotopes of hydrogen include ²H (deuterium) and ³H (tritium). Isotopes of carbon include ¹¹C, ¹³C, and ¹⁴C. Isotopes of nitrogen include ¹³N and ¹⁵N. Isotopes of oxygen include ¹⁵O, ¹⁷O, and ¹⁸O. An isotope of fluorine includes ¹⁸F. An isotope of sulfur includes ³⁵S. An isotope of chlorine includes ³⁶Cl. Isotopes of bromine include ⁷⁵Br, ⁷⁶Br, ⁷⁷Br, and ⁸²Br. Isotopes of iodine include ¹²³I, ¹²⁴I, ¹²⁵I, and ¹³¹I. Also provided are pharmaceutical compositions comprising a compound or a pharmaceutically acceptable salt thereof described herein, wherein the naturally occurring distribution of the isotopes in the pharmaceutical composition is perturbed. Also provided are pharmaceutical compositions comprising a compound or a pharmaceutically acceptable salt thereof described herein enriched at one or more positions with an isotope other than the most naturally abundant isotope. Methods are readily available to measure such isotope perturbations or enrichments, such as, mass spectrometry, and for isotopes that are radio-isotopes additional methods are available, such as, radio-detectors used in connection with HPLC or GC. Certain isotopically-labeled compounds and pharmaceutically acceptable salts thereof described herein are useful in compound and/or substrate tissue distribution assays. In some embodiments the radionuclide ³H and/or ¹⁴C isotopes are useful in these studies. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds and pharmaceutically acceptable salts thereof described herein can generally be prepared by following procedures analogous to those disclosed in the Examples infra, by substituting an isotopically labeled reagent for a non-isotopically labeled reagent. Moreover, it should be understood that all of the atoms represented in the compounds and pharmaceutically acceptable salts thereof described herein can be either the most commonly occurring isotope of such atoms or a scarcer radio-isotope or nonradioactive isotope.

In carrying out the procedures of the methods described herein, it is of course to be understood that reference to particular buffers, media, reagents, cells, culture conditions and the like are not intended to be limiting, but are to be read so as to include all related materials that one of ordinary skill in the art would recognize as being of interest or value in the particular context in which that discussion is presented. For example, it is often possible to substitute one buffer system or culture medium for another and still achieve similar, if not identical, results. Those of skill in the art will have sufficient knowledge of such systems and methodologies so as to be able, without undue experimentation, to make such substitutions as will optimally serve their purposes in using the methods and procedures disclosed herein.

### EXAMPLES

The compounds, or pharmaceutically acceptable salts thereof, compositions, and methods described herein are further illustrated by the following non-limiting examples.

As used herein, the following abbreviations have the following meanings. If an abbreviation is not defined, it has its generally accepted meaning.

### ABBREVIATIONS

d: Doublet
dd: Doublet of doublets
DCM: Dichloromethane
DME: Dimethoxyethane
DMF: Dimethylformamide
DMSO: Dimethylsulfoxide
ES+: Electrospray Positive Ionisation
ES−: Electrospray Negative Ionisation
Et₂O: Diethyl ether
EtOAc: Ethyl acetate
g: Gram
h: Hour
HPLC: High Performance Liquid Chromatography
Hz: Hertz
J: Coupling constant
LCMS: Liquid Chromatography Mass Spectrometry
LHMDS: Lithium bis(trimethylsilyl)amide
m: Multiplet
M: Mass
MeCN: Acetonitrile
MeOH: Methanol
mg: Milligram
MHz: Megahertz
mL: Milliliter
mmol: Millimole
NBS: N-bromosuccinimide
Pd/C: Palladium on carbon
Pd(dppf)Cl₂.CH₂Cl₂: [1,1′-Bis(diphenylphosphino)ferrocene]dichloropalladium(II)].CH₂Cl₂
Pd(PPh₃)₄: Tetrakis(triphenylphosphine)palladium(0)
RT: Retention time
q: Quartet
qn: Quintet
r.t.: Room temperature
s: Singlet
THF: Tetrahydrofuran
v/v: Volume to volume
w/v: Weight to volume

| Analytical condition | Method | Description | |
|---|---|---|---|
| 15cm_Formic_Ascentis_HPLC_MeCN | 1 | Solvents: | Acetonitrile (far UV grade) with 0.1% (v/v) formic |
| | | | acid. Water (high purity via PureLab ® Ultra unit) with |
| | | | 0.1% formic acid |
| | | Column: | Supelco, Ascentis ® Express C18 or |
| | | | Hichrom Halo C18, 2.7 μm C18, 150 × 4.6 mm |
| | | Flow Rate: | 1 mL/min |
| | | gradient: | A: Water/formic acid |
| | | | B: MeCN/formic acid |

| | | |
|---|---|---|
| Time | A % | B % |
| 0.00 | 96 | 4 |
| 3.00 | 96 | 4 |
| 9.00 | 0 | 100 |
| 13.6 | 0 | 100 |
| 13.7 | 96 | 4 |
| 15 | 96 | 4 |

| | | Typical injections 2-7 μL (concentration ~0.2-1.0 mg/mL) | |
|---|---|---|---|
| 10cm_ESCI_formic_MeCN | 2 | Solvents: | Acetonitrile (far UV grade) with 0.1% (v/v) formic |
| | | | acid. Water (high purity via PureLab ® Ultra unit) |
| | | | with 0.1% formic acid |
| | | Column: | Phenomenex Luna ® 5 μm C18 (2), 100 × 4.6 mm |
| | | | (Plus guard cartridge) |
| | | Flow Rate: | 2 mL/min |
| | | gradient: | A: Water/formic acid |
| | | | B: MeCN/formic acid |

| | | |
|---|---|---|
| Time | A % | B % |
| 0.00 | 95 | 5 |
| 3.50 | 5 | 95 |
| 5.50 | 5 | 95 |
| 5.60 | 95 | 5 |
| 6.50 | 95 | 5 |

| | | Typical injections 2-7 μL (concentration ~0.2-1.0 mg/mL) | |
|---|---|---|---|
| 10cm_Formic_ACE- | 3 | Solvents: | Acetonitrile (far UV grade) with 0.1% (v/v) formic |
| AR_HPLC_MeCN | | | acid. Water (high purity via PureLab ® Ultra unit) |
| | | | with 0.1% formic acid |
| | | Column: | Hichrom ACE 3 C18-AR mixed mode column 100 × |
| | | | 4.6 mm |
| | | Flow Rate: | 1 mL/min |
| | | gradient: | A: Water/formic acid |
| | | | B: MeCN/formic acid |

| | | |
|---|---|---|
| Time | A % | B % |
| 0.00 | 98 | 2 |
| 3.00 | 98 | 2 |
| 12.00 | 0 | 100 |
| 15.4 | 0 | 100 |
| 15.5 | 98 | 2 |
| 17 | 98 | 2 |

Typical injections 0.2-10 μL

### Synthetic Section

### General Methods

### Method A: Hydroxamic Acid Formation

A solution of ester (1 equiv), hydroxylamine (50% w/v in water, 10 equiv) and NaOH (3.75 M in water, 3 equiv) in 1:1 v/v THF:MeOH (4 mL per mmol of ester) was stirred at 20° C. for 1.5 h.

### Method B: Suzuki Reaction

A suspension of aryl or vinyl halide (1 equiv), boronic acid/ester (1.3 equiv), Pd(PPh₃)₄ (0.05 equiv) and CsF (1.5 equiv) in 3:1 v/v DME:MeOH (6 mL per mmol of halide) was stirred in a microwave reactor at 120° C. for 30-60 min, until LCMS analysis indicated reaction was complete. The supernatant was decanted and evaporated to dryness.

### Method C: Chan-Lam Coupling

A suspension of pyrazole (1 equiv), boronic acid (2 equiv), Cu(OAc)₂ (1.5 equiv), dry pyridine (2 equiv) and 4 Å molecular sieves (150 mg per mmol of pyrazole) in dry CH₂Cl₂ (1.7 mL per mmol of pyrazole) was stirred vigorously in air at 20° C. for 19 h. Solids were removed by filtration through Celite®, washing with CH₂Cl₂. The filtrate was concentrated under vacuum.

### Method D: Preparation of Lithium Enolates

A stirred solution of LHMDS (1.0 M in THF, 1 equiv) held at −78° C. under nitrogen, was treated dropwise with a solution of ketone (1 equiv) in dry Et₂O (0.8 mL per mmol ketone). After stirring at −78° C. for 40 min, a solution of diethyl oxalate (1 equiv) in dry Et₂O (0.25 mL per mmol ketone) was added and the reaction was allowed to warm to 20° C. over 16 h. The solution was concentrated under vacuum.

### Method E: Cyclisation Reaction Between Hydrazines and Lithium Enolates

A stirred suspension of lithium enolate (1 equiv) in acetic acid (1.5 mL per mmol of enolate) was treated with hydrazine (1.1 equiv) at 0° C. Once addition was complete, the reaction mixture was stirred in a sealed tube at 120° C. for 8 h. After cooling to room temperature, the mixture was concentrated under vacuum.

### Preparation of Intermediate 1

### Methyl 3-bromo-1-phenyl-1H-pyrazole-5-carboxylate

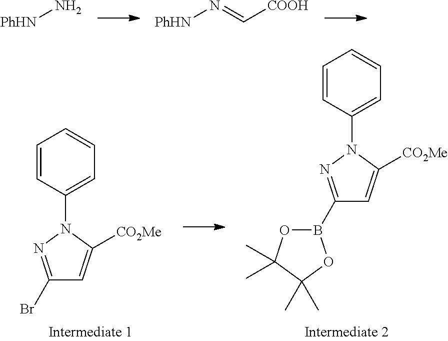

### Step 1: 2-(Phenylhydrazono)acetic acid

A stirred solution of phenylhydrazine (8.7 mL, 88.5 mmol) in water (250 mL) at 0° C. was treated sequentially with concentrated HCl (9.8 mL) and glyoxylic acid (50% w/v in water, 10.8 mL, 97.0 mmol). After 1 h stirring at 20° C., the precipitate was collected by filtration, washing with water (3×20 mL). The solid was dissolved in EtOAc-MeOH (1:1 v/v, 400 mL), dried (Na₂SO₄) and concentrated under vacuum to yield the title compound as a ˜95:5 mixture of (E) and (Z) isomers as a yellow powder (13.7 g, 94%). LCMS (ES−) 163 (M−H)⁻.

### Step 2: Methyl 3-bromo-1-phenyl-1H-pyrazole-5-carboxylate (Intermediate 1)

A stirred solution of 2-(phenylhydrazono)acetic acid (5.00 g, 30.5 mmol) in DMF (60 mL) at 0° C. was treated with a solution of NBS (10.8 g, 60.7 mmol) in DMF (60 mL) over 10 min. The resulting mixture was stirred at 20° C. for 20 min before cooling to 0° C. Methyl propiolate (13.6 mL, 152 mmol) was added, followed by triethylamine (4.3 mL, 30.9 mmol) and the reaction mixture stirred at 20° C. for 2 h. After this time the mixture was poured into water (300 mL) and extracted with Et₂O (3×100 mL). The combined organic extracts were washed with water (2×50 mL) and brine (50 mL), dried (Na₂SO₄) and concentrated under vacuum. The residue was dissolved in the minimum volume of toluene, then treated with iso-hexane until the solution became cloudy. On standing a solid formed, which was removed by filtration. The filtrate was concentrated and purified by silica gel chromatography (gradient elution i-hexane to 100% EtOAc in i-hexane) to yield the title compound as a pale brown powder (1.62 g, 19%). LCMS (ES+) 281/283 (M+H)⁺; ¹H NMR δ (ppm) (400 MHz, CDCl₃): 7.49-7.44 (3H, m), 7.43-7.39 (2H, m), 7.00 (1H, s), 3.80 (3H, s).

### Preparation of Intermediate 2

### Methyl 1-phenyl-3-(4,4,5,5-tetramethyl-1,3,2-dioxaborolan-2-yl)-1H-pyrazole-5-carboxylate

A suspension of intermediate 1 (1.62 g, 5.76 mmol), bis(pinacolato)diboron (2.23 g, 8.78 mmol), Pd(dppf)Cl₂.CH₂Cl₂ (714 mg, 0.87 mmol) and potassium acetate (1.74 g, 17.7 mmol) in dry dioxane (32 mL) was purged with nitrogen and stirred at reflux for 2 h. The title compound constituted 95% of the crude mixture, as judged by LCMS. The crude solution was stored at 20° C. under N₂ and used without purification.

### Preparation of Intermediate 3

### Ethyl 3-(2-methylphenyl)-1H-pyrazole-5-carboxylate

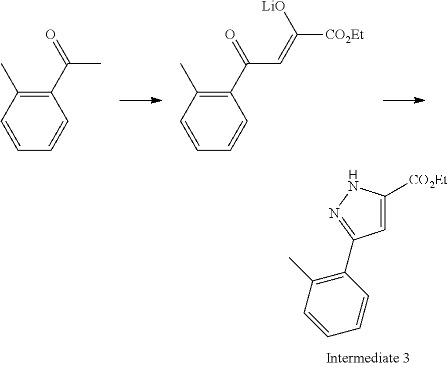

### Step 1: [(Z)-1-Ethoxycarbonyl-3-(2-methylphenyl)-3-oxo-prop-1-enoxy]lithium

Following method D from 1-(2-methyl-phenyl)ethanone (1.00 g, 7.45 mmol). Yellow powder (1.82 g) was obtained and used without further purification. LCMS (ES−) 233 (M-Li)⁻.

### Step 2: Ethyl 3-(2-methylphenyl)-1H-pyrazole-5-carboxylate (Intermediate 3)

Following method E from [(Z)-1-ethoxycarbonyl-3-(2-methyl-phenyl)-3-oxo-prop-1-enoxy]lithium (503 mg, 2.09 mmol) and hydrazine (35% w/v in water, 0.20 mL, 2.25 mmol). Purification by silica gel chromatography (gradient elution i-hexane to 100% EtOAc in i-hexane) gave the title compound (415 mg, 88% over two steps). LCMS (ES+) 231 (M+H)⁺. ¹H NMR δ (ppm) (400 MHz, CDCl₃): 10.66 (1H, s), 7.45 (1H, d, J=7.3 Hz), 7.34-7.27 (3H, m), 6.97 (1H, s), 4.43 (2H, q, J=7.2 Hz), 2.44 (3H, s), 1.42 (3H, t, J=7.2 Hz).

### Preparation of Intermediate 4

### Methyl 4-bromo-1-phenyl-pyrrole-2-carboxylate

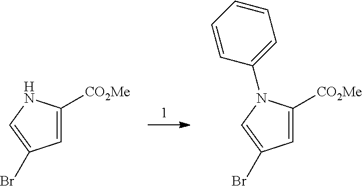 [SMILES: COC(=O)c1cc(Br)c[nH]1 ; COC(=O)c1cc(Br)cn1-c1ccccc1]

Following method C from methyl 4-bromopyrrole-2-carboxylate (4.01 g, 19.7 mmol) and phenylboronic acid (4.86 g, 39.9 mmol) Purification by silica gel chromatography (gradient elution i-hexane to 100% EtOAc in i-hexane) yielded a mixed fraction that partially crystallised on standing at 20° C. The precipitate was collected by filtration, washing with i-hexane (10×5 mL) to yield the title compound as white prisms (2.48 g, 45%). LCMS (ES+) 280/282 (M+H)⁺.

### EXAMPLES

### Example 1

### N-Hydroxy-1,3-diphenylpyrazole-5-carboxamide

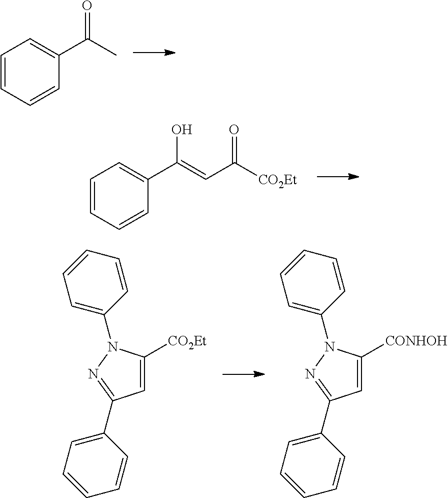

### Step 1: (Z)-Ethyl-4-hydroxy-2-oxo-4-phenylbut-3-enoate

A stirred solution of sodium (3 mL, 30% dispersion in toluene) was treated with sufficient EtOH to dissolve all traces of metal. The mixture was allowed to cool to room temperature and diethyl oxalate (5.8 mL, 43.0 mmol) added dropwise, followed by acetophenone (5 mL, 43.0 mmol), before stirring at 20° C. for 17 h. AcOH (25 mL), water (25 mL) and Et₂O (50 mL) were added and the biphasic mixture stirred vigorously. The aqueous layer was separated and extracted with Et₂O (3×50 mL). The combined organic layers were dried (MgSO₄), filtered and concentrated. The resulting oil was cooled (−78° C.) and the precipitated solid recrystallized from hot MeOH to give the title compound as a white crystalline solid (2.97 g, 31%). ¹H NMR δ (ppm) (400 MHz, CDCl₃): 15.31 (1H, s), 8.02-7.99 (2H, m), 7.64-7.59 (1H, m), 7.51 (2H, apparent t, J=7.6 Hz), 7.09 (1H, s), 4.41 (2H, q, J=7.2 Hz), 1.42 (3H, t, J=7.3 Hz).

### Step 2: Ethyl 1,3-diphenylpyrazole-5-carboxylate

A stirred solution of (Z)-ethyl-4-hydroxy-2-oxo-4-phenylbut-3-enoate (1 g, 4.55 mmol) in EtOH (40 mL) was treated dropwise with phenylhydrazine (0.54 mL, 5.45 mmol), and the mixture heated to 80° C. for 3 h. The mixture was concentrated and purified by silica gel chromatography (gradient elution i-hexane to 10% EtOAc in i-hexane). The products isolated from the column appeared to be non-cyclized intermediates, which were redissolved in EtOH and treated with concentrated H₂SO₄ (1 mL). The reaction was stirred at 80° C. for 2 h, concentrated and partitioned between CH₂Cl₂ and water. After vigorous shaking, the organics were passed through a phase separator and concentrated to give the title compound as a white solid (80 mg, 6%) as the minor regioisomeric product of the reaction. LCMS (ES+) 293 (M+H)⁺.

### Step 3: N-Hydroxy-1,3-diphenylpyrazole-5-carboxamide

Following method A from ethyl 1,3-diphenylpyrazole-5-carboxylate (35 mg, 0.12 mmol). The reaction was neutralized with aqueous 1M HCl, extracted into EtOAc (2×30 mL) and the organics washed with water (30 mL) and brine (30 mL). The organic phase was dried (MgSO₄), filtered and concentrated, and the resulting solid recrystallized from CH₂Cl₂ to give the title compound as a white solid (1 mg, 3%). LCMS (ES+) 280 (M+H)⁺, RT 8.48 min (Analytical method 1); ¹H NMR δ (ppm) (400 MHz, DMSO-d₆): 7.91 (2H, d, J=7.1 Hz), 7.60 (2H, d, J=7.8 Hz), 7.55-7.46 (4H, m), 7.45-7.38 (2H, m), 7.13 (1H, s), OH and NH not observed.

### Example 2

### N-Hydroxy-3-(benzothiophen-7-yl)-1-phenyl-1H-pyrazole-5-carboxamide

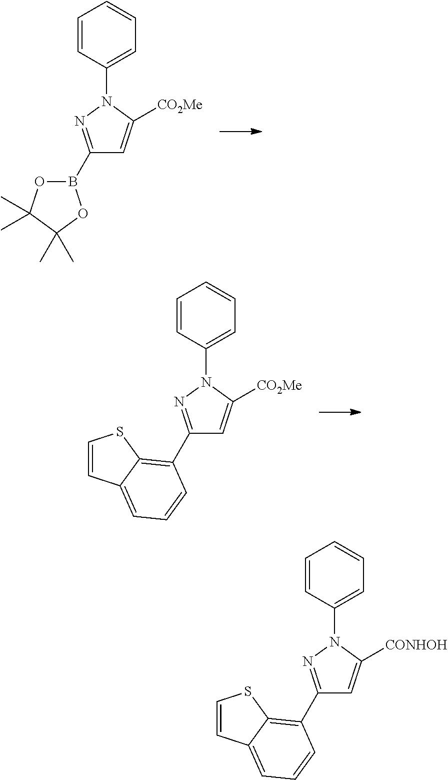

### Step 1: Methyl 3-(benzothiophen-7-yl)-1-phenyl-pyrazole-5-carboxylate

A suspension of intermediate 2 (3 mL of the solution from step 3, ˜0.54 mmol), 7-bromobenzothiophene (116 mg, 0.54 mmol), Pd(PPh₃)₄ (32.1 mg, 28 μmol), caesium fluoride (134 mg, 0.88 mmol) and methanol (1 mL) was stirred in a microwave reactor at 120° C. for 30 min. The supernatant was decanted, evaporated to dryness and used immediately.

### Step 2: N-Hydroxy-3-(benzothiophen-7-yl)-1-phenyl-1H-pyrazole-5-carboxamide

Following method A from methyl 3-(benzothiophen-7-yl)-1-phenyl-pyrazole-5-carboxylate (˜181 mg, 0.54 mmol). Purification by preparative HPLC gave the title compound (40 mg, 22% over three steps). LCMS (ES+) 336 (M+H)⁺, RT 3.82 min (Analytical method 2); ¹H NMR δ (ppm) (400 MHz, DMSO-d₆): 11.52 (1H, s), 9.47 (1H, s), 8.01-7.95 (2H, m), 7.88 (1H, d, J=5.4 Hz), 7.70-7.68 (2H, m), 7.63-7.47 (6H, m).

### Example 3

### N-Hydroxy-3-(2-methylphenyl)-1-phenyl-1H-pyrazole-5-carboxamide

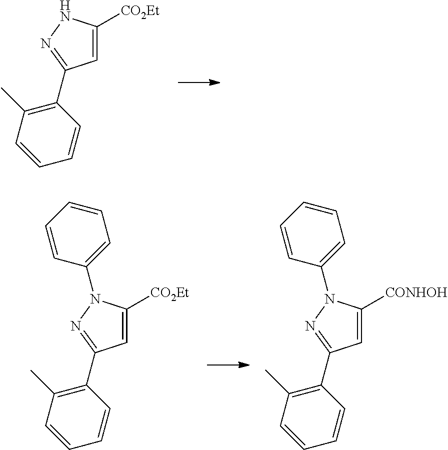

### Step 1: Ethyl 3-(2-methylphenyl)-1-phenyl-1H-pyrazole-5-carboxylate

Following method C from intermediate 3 (200 mg, 0.87 mmol) and phenylboronic acid (213 mg, 1.75 mmol). Purification by silica gel chromatography (gradient elution i-hexane to 50% EtOAc in i-hexane) gave the title compound (211 mg, 79%). LCMS (ES+) 307 (M+H)⁺.

### Step 2: N-Hydroxy-3-(2-methylphenyl)-1-phenyl-1H-pyrazole-5-carboxamide

Following method A from ethyl 3-(2-methyl-phenyl)-1-phenyl-1H-pyrazole-5-carboxylate (211 mg, 0.69 mmol) Purification by preparative HPLC gave the title compound (115 mg, 57%). LCMS (ES+) 294 (M+H)⁺, RT 3.57 min. (Analytical method 2); ¹H NMR δ (ppm) (400 MHz, DMF-d₇): 11.53 (1H, s), 9.72 (1H, s), 7.74-7.71 (1H, m), 7.67-7.63 (2H, m), 7.57-7.50 (2H, m), 7.48-7.43 (1H, m), 7.38-7.31 (3H, m), 7.15 (1H, s), 2.58 (3H, s).

### Example 4

### N-Hydroxy-3-benzyl-1-phenyl-1H-pyrazole-5-carboxamide

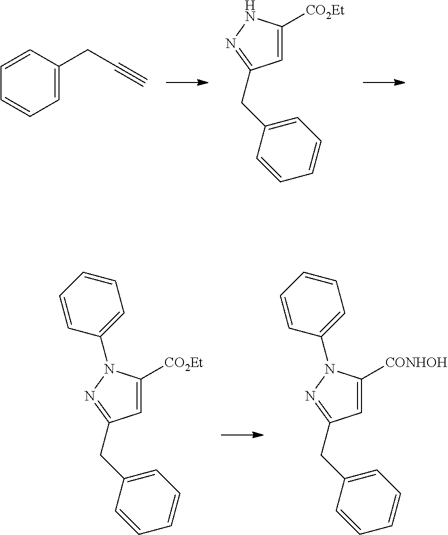

### Step 1: Ethyl 3-benzyl-1H-pyrazole-5-carboxylate

A stirred solution of 3-phenyl-1-propyne (359 mg, 3.09 mmol) in dry THF (12 mL) held at −78° C. under N₂ was treated with n-butyllithium (1.6 M in hexanes, 1.93 mL, 3.09 mmol) over 10 min. The resulting red solution was stirred at −78° C. for 2 h before being added to a stirred suspension of CuCN.6LiCl (prepared by stirring CuCN and LiCl in a 1:6 molar ratio under vacuum at 160° C. for 10 h, 1.07 g, 3.11 mmol) in dry THF (18 mL) held at −78° C. under N₂. The reaction was warmed to −5° C. and stirred for 1 h before a solution of ethyl diazoacetate (0.33 mL, 3.12 mmol) in dry THF (12 mL) was added in 10 portions over 2 min. Gas evolution was observed. The mixture was stirred at 20° C. for 21 h, then quenched with saturated aqueous NH₄Cl (30 mL). After a further 1 h stirring at 20° C., the mixture was extracted with Et₂O (3×50 mL); the combined organic extracts were washed with brine (30 mL), dried (Na₂SO₄) and concentrated under vacuum. The brown liquid residue was purified by silica gel chromatography (gradient elution i-hexane to 100% EtOAc in i-hexane) to yield the title compound as a brown oil (63% purity as judged by LCMS), which was used without further purification.

### Step 2: Ethyl 3-benzyl-1-phenyl-pyrazole-5-carboxylate

Following method C from ethyl 3-benzyl-1H-pyrazole-5-carboxylate (230 mg of the mixture from the previous step, 1.00 mmol) and phenylboronic acid (251 mg, 2.06 mmol). Purification by silica gel chromatography (gradient elution i-hexane to 100% EtOAc in i-hexane) gave the title compound (152 mg, 16% over two steps). LCMS (ES+) 307 (M+H)⁺.

### Step 3: N-Hydroxy-3-benzyl-1-phenyl-1H-pyrazole-5-carboxamide

Following method A from ethyl 3-benzyl-1-phenyl-pyrazole-5-carboxylate (152 mg, 0.50 mmol). Purification by preparative HPLC gave the title compound (71 mg, 49%). LCMS (ES+) 294 (M+H)⁺, RT 3.34 min (Analytical method 2); ¹H NMR δ (ppm) (400 MHz, DMSO-d₆): 11.29 (1H, s), 9.28 (1H, s), 7.52-7.23 (10H, m), 6.56 (1H, s), 4.01 (2H, s).

### Example 5

### N-Hydroxy-3-(2-chlorophenyl)-1-phenyl-1H-pyrazole-5-carboxamide

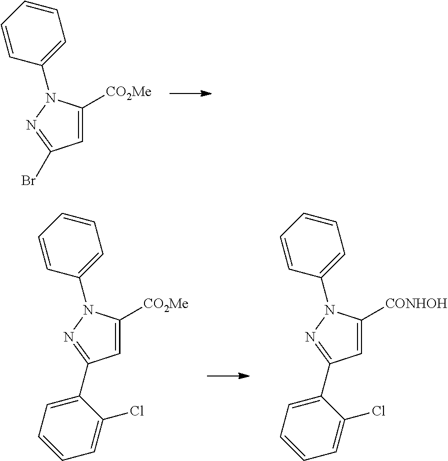

### Step 1: Methyl 3-(2-chlorophenyl)-1-phenyl-1H-pyrazole-5-carboxylate

Following method B from intermediate 1 (298 mg, 1.06 mmol) and 2-chlorophenylboronic acid (226 mg, 1.45 mmol). Material used immediately without characterisation.

### Step 2: N-Hydroxy-3-(2-chlorophenyl)-1-phenyl-1H-pyrazole-5-carboxamide

Following method A from methyl 3-(2-chlorophenyl)-1-phenyl-1H-pyrazole-5-carboxylate (˜332 mg, 1.06 mmol). Purification by preparative HPLC gave the title compound (78 mg, 23% over two steps). LCMS (ES+) 314/316 (M+H)⁺, RT 3.60 min (Analytical method 2); ¹H NMR δ (ppm) (400 MHz, DMSO-d₆): 11.48 (1H, s), 9.42 (1H, s), 7.91-7.87 (1H, m), 7.65-7.46 (8H, m), 7.28 (1H, s).

### Example 6

### N-Hydroxy-3-(cyclopenten-1-yl)-1-phenyl-1H-pyrazole-5-carboxamide

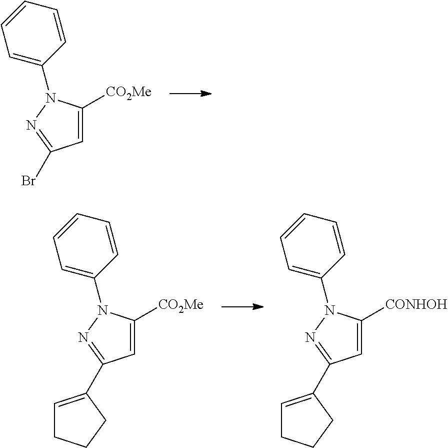

### Step 1: Methyl 3-(cyclopenten-1-yl)-1-phenyl-1H-pyrazole-5-carboxylate

Following method B from intermediate 1 (201 mg, 0.72 mmol) and 2-(cyclopenten-1-yl)-4,4,5,5-tetramethyl-1,3,2-dioxaborolane (176 mg, 0.91 mmol) Purification by silica gel chromatography (gradient elution i-hexane to 100% EtOAc in i-hexane) gave the title compound as a pale yellow liquid (98 mg, 51%). LCMS (ES+) 269 (M+H)⁺. ¹H NMR δ (ppm) (400 MHz, CDCl₃): 7.48-7.38 (5H, m), 7.06 (1H, s), 6.29-6.24 (1H, m), 3.78 (3H, s), 2.80-2.72 (2H, m), 2.58-2.49 (2H, m), 2.06-1.97 (2H, qn, J=7.5 Hz).

### Step 2: N-Hydroxy-3-(cyclopenten-1-yl)-1-phenyl-1H-pyrazole-5-carboxamide

Following method A from methyl 3-(cyclopenten-1-yl)-1-phenyl-1H-pyrazole-5-carboxylate (43 mg, 0.16 mmol). Purification by preparative HPLC gave the title compound (21 mg, 49%). LCMS (ES+) 270 (M+H)⁺, RT 3.44 min (Analytical method 2); ¹H NMR δ (ppm) (400 MHz, DMSO-d₆): 11.36 (1H, s), 9.35 (1H, s), 7.54-7.39 (5H, m), 6.94 (1H, s), 6.30 (1H, t, J=2.1 Hz), 2.74-2.67 (2H, m), 2.03-1.94 (2H, m), 2 protons obscured by solvent peak.

### Example 7

### N-Hydroxy-3-cyclopentyl-1-phenyl-1H-pyrazole-5-carboxamide

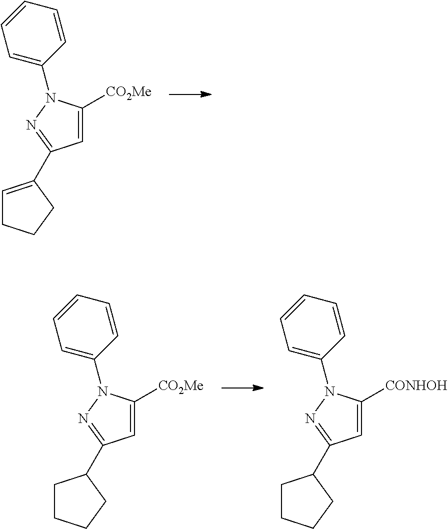

### Step 1: Methyl 3-cyclopentyl-1-phenyl-1H-pyrazole-5-carboxylate

A suspension of methyl 3-(cyclopenten-1-yl)-1-phenyl-1H-pyrazole-5-carboxylate (96 mg, 0.36 mmol) and 5% Pd/C-water paste (104 mg) in EtOH (10 mL) was stirred at 20° C. under 1.7 bar H₂ pressure for 3.5 h. The mixture was filtered through Celite® and the filtrate concentrated to give the title compound as a pale yellow liquid (84 mg, 87%). LCMS (ES+) 271 (M+H)⁺.

### Step 2: N-Hydroxy-3-cyclopentyl-1-phenyl-1H-pyrazole-5-carboxamide

Following method A from methyl 3-cyclopentyl-1-phenyl-1H-pyrazole-5-carboxylate (84 mg, 0.31 mmol). Purification by preparative HPLC gave the title compound (43 mg, 51%). LCMS (ES+) 272 (M+H)⁺, RT 3.43 min (Analytical method 2); ¹H NMR δ (ppm) (400 MHz, DMSO-d₆): 11.29 (1H, s), 9.31 (1H, s), 7.51-7.37 (5H, m), 6.63 (1H, s), 3.12 (1H, qn, J=7.7 Hz), 2.08-1.99 (2H, m), 1.80-1.63 (6H, m).

### Example 8

### N-Hydroxy-3-(5-chloro-3-pyridyl)-1-phenyl-1H-pyrazole-3-carboxamide

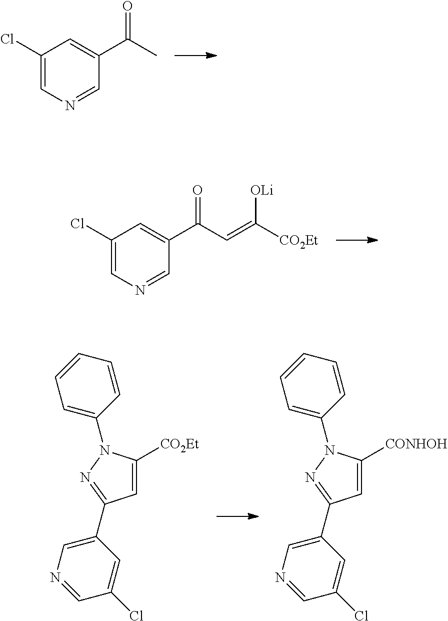

### Step 1: [(Z)-3-(5-Chloro-3-pyridyl)-1-ethoxycarbonyl-3-oxo-prop-1-enoxy]lithium

Following method D from 1-(5-chloro-3-pyridyl)ethanone (1.03 g). The title compound was obtained as a beige solid (1.39 g, 80%). LCMS (ES−) 254/256 (M-Li)⁻.

### Step 2: Ethyl 3-(5-chloro-3-pyridyl)-1-phenyl-1H-pyrazole-5-carboxylate

A suspension of [(Z)-3-(5-chloro-3-pyridyl)-1-ethoxycarbonyl-3-oxo-prop-1-enoxy]lithium (650 mg, 2.48 mmol) in acetic acid (3.3 mL) was treated with phenylhydrazine (0.24 mL, 2.44 mmol) at 20° C. The reaction mixture was stirred at 100° C. in a sealed tube for 1 h. After cooling to room temperature the mixture was diluted with CH₂Cl₂ (20 mL), washed with water (20 mL), NaOH (2.5 M in water, 20 mL) and further water (20 mL), dried (Na₂SO₄) and concentrated under vacuum. Purification by silica gel chromatography (gradient elution i-hexane to 100% EtOAc in i-hexane) gave the title compound as a yellow powder (53 mg, 7%) as the minor regioisomeric product. LCMS (ES+) 328/330 (M+H)⁺.

### Step 3: N-Hydroxy-3-(5-chloro-3-pyridyl)-1-phenyl-1H-pyrazole-3-carboxamide

Following method A from ethyl 3-(5-chloro-3-pyridyl)-1-phenyl-1H-pyrazole-5-carboxylate (53 mg, 0.16 mmol). Purification by preparative HPLC gave the title compound (18 mg, 35%). LCMS (ES+) 315/317 (M+H)⁺, RT 9.55 min (Analytical method 3); ¹H NMR δ (ppm) (400 MHz, DMSO-d₆): 11.48 (1H, s), 9.48 (1H, s), 9.10 (1H, d, J=1.8 Hz), 8.68 (1H, d, J=2.3 Hz), 8.41 (1H, dd, J=2.1, 2.1 Hz), 7.61-7.47 (5H, m), 7.45 (1H, s).

### Example 9

### N-Hydroxy-3-(2-methyl-3-pyridyl)-1-phenyl-1H-pyrazole-5-carboxamide

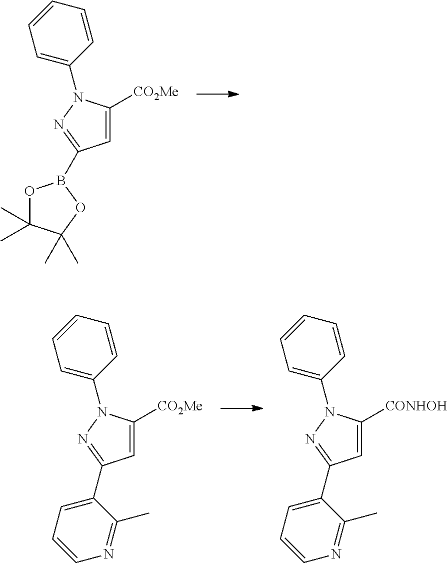

### Step 1: Methyl 3-(2-methyl-3-pyridyl)-1-phenyl-1H-pyrazole-5-carboxylate

A suspension of intermediate 2 (3 mL of the solution, ˜0.54 mmol), 3-bromo-2-methylpyridine (127 mg, 0.74 mmol), Pd(PPh₃)₄ (35.7 mg, 31 μmol), caesium fluoride (127 mg, 0.84 mmol) and methanol (1 mL) was stirred in a microwave reactor at 120° C. for 30 min. The supernatant was decanted, evaporated to dryness and used immediately.

### Step 2: N-Hydroxy-3-(2-methyl-3-pyridyl)-1-phenyl-1H-pyrazole-5-carboxamide

Following method A from methyl 3-(2-methyl-3-pyridyl)-1-phenyl-1H-pyrazole-5-carboxylate (˜158 mg, 0.54 mmol). Purification by preparative HPLC gave the title compound (33 mg, 21% over three steps from intermediate 1). LCMS (ES+) 295 (M+H)⁺, RT 7.25 min (Analytical method 3); ¹H NMR δ (ppm) (400 MHz, DMSO-d₆): 11.48 (1H, s), 9.44 (1H, s), 8.51 (1H, dd, J=1.7, 4.7 Hz), 8.05 (1H, dd, J=1.6, 7.8 Hz), 7.60-7.51 (4H, m), 7.50-7.45 (1H, m), 7.37 (1H, dd, J=4.7, 7.8 Hz), 7.18 (1H, s), 2.76 (3H, s).

### Example 10

### N-hydroxy-1-(3-fluoro-2-methyl-phenyl)-3-(2-methyl-phenyl)-1H-pyrazole-5-carboxamide

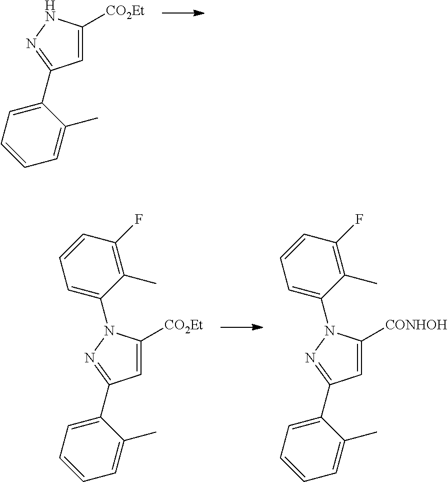

### Step 1: Ethyl 1-(3-fluoro-2-methyl-phenyl)-3-(2-methyl-phenyl)-1H-pyrazole-5-carboxylate

Following method C from intermediate 3 (200 mg, 0.87 mmol) and 3-fluoro-2-methyl-phenylboronic acid (269 mg, 1.75 mmol). Purification by silica gel chromatography (gradient elution i-hexane to 100% EtOAc in i-hexane) gave the title compound as a yellow liquid (193 mg, 66%). LCMS (ES+) 339 (M+H)⁺.

### Step 2: N-hydroxy-1-(3-fluoro-2-methyl-phenyl)-3-(2-methyl-phenyl)-1H-pyrazole-5-carboxamide

Following method A from ethyl 1-(3-fluoro-2-methyl-phenyl)-3-(2-methyl-phenyl)-1H-pyrazole-5-carboxylate (180 mg, 0.53 mmol). Purification by silica gel chromatography (gradient elution CH₂Cl₂ to 10% MeOH in CH₂Cl₂) gave the title compound as a white solid (36 mg, 21%). LCMS (ES+) 326 (M+H)⁺, RT 3.84 min (Analytical method 2); ¹H NMR δ (ppm) (400 MHz, DMSO-d₆): 11.41 (1H, s), 9.29 (1H, s), 7.64-7.60 (1H, m), 7.41-7.30 (5H, m), 7.24-7.19 (1H, m), 7.16 (1H, s), 2.52 (3H, s), 1.98 (3H, d, J=1.9 Hz).

### Example 11

### N-Hydroxy-3-(1-methyl-3,6-dihydro-2H-pyridin-4-yl)-1-phenyl-1H-pyrazole-5-carboxamide

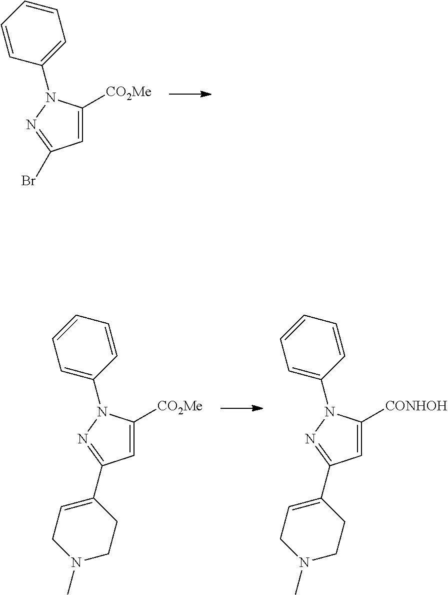

### Step 1: Methyl 3-(1-methyl-3,6-dihydro-2H-pyridin-4-yl)-1-phenyl-1H-pyrazole-5-carboxylate

Following method B from intermediate 1 (143 mg, 0.51 mmol) and 1-methyl-4-(4,4,5,5-tetramethyl-1,3,2-dioxaborolan-2-yl)-3,6-dihydro-2H-pyridine (144 mg, 0.65 mmol) Material used immediately without characterisation.

### Step 2: N-Hydroxy-3-(1-methyl-3,6-dihydro-2H-pyridin-4-yl)-1-phenyl-1H-pyrazole-5-carboxamide

Following method A from methyl 3-(1-methyl-3,6-dihydro-2H-pyridin-4-yl)-1-phenyl-1H-pyrazole-5-carboxylate (˜152 mg, 0.51 mmol). Purification by preparative HPLC gave the title compound (26 mg, 17% over two steps). LCMS (ES+) 299 (M+H)⁺, RT 6.97 min (Analytical method 3); ¹H NMR δ (ppm) (400 MHz, DMSO-d₆): 8.51 (1H, s, NH), 7.51-7.47 (5H, m), 6.89 (1H, s), 6.33 (1H, t, J=3.3 Hz), 3.05 (2H, d, J=2.9 Hz), 2.31 (3H, s), OH not observed, 4 protons obscured by solvent peak.

### Example 12

### N-Hydroxy-1,3-bis(2-methylphenyl)-1H-pyrazole-5-carboxamide

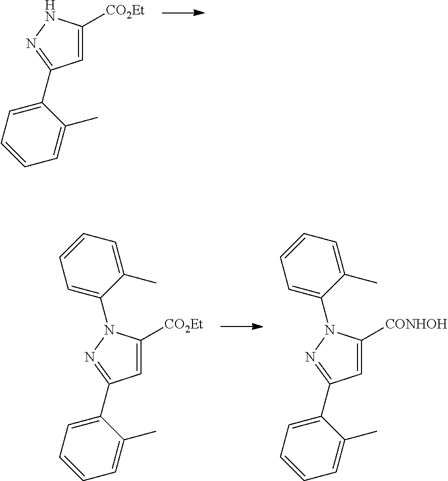

### Step 1: Ethyl 1,3-bis(2-methylphenyl)-1H-pyrazole-5-carboxylate

Following method C from intermediate 3 (171 mg, 0.74 mmol) and 2-methylphenylboronic acid (204 mg, 1.50 mmol). Purification by silica gel chromatography (gradient elution i-hexane to 100% EtOAc in i-hexane) gave the title compound as a colourless liquid (151 mg, 63%). LCMS (ES+) 321 (M+H)⁺.

### Step 2: N-Hydroxy-1,3-bis(2-methylphenyl)-1H-pyrazole-5-carboxamide

Following method A from ethyl 1,3-bis(2-methylphenyl)-1H-pyrazole-5-carboxylate (144 mg, 0.45 mmol) Purification by preparative HPLC gave the title compound (80 mg, 58%). LCMS (ES+) 308 (M+H)⁺, RT 3.72 min (Analytical method 2); ¹H NMR δ (ppm) (400 MHz, DMSO-d₆): 11.35 (1H, s), 9.29 (1H, s), 7.64-7.60 (1H, m), 7.43-7.38 (2H, m), 7.35-7.29 (5H, m), 7.12 (1H, s), 2.52 (3H, s), 2.08 (3H, s).

### Example 13

### N-Hydroxy-3-benzyloxy-1-phenyl-1H-pyrazole-5-carboxamide

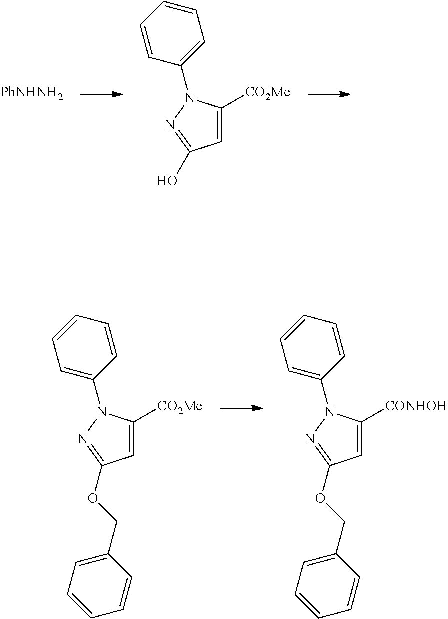

### Step 1: Methyl 3-hydroxy-1-phenyl-1H-pyrazole-5-carboxylate

A stirred solution of dimethyl but-2-ynedioate (1.37 mL, 11.2 mmol) in toluene (8 mL) and acetic acid (8 mL) at 0° C. was treated cautiously with phenylhydrazine (1.00 mL, 10.2 mmol). The mixture was stirred at 20° C. for 1 h, then heated to reflux for 4 h. On standing at 20° C. for 4 days a white precipitate formed which was collected by filtration, washing with Et₂O (3×10 mL), yielding the title compound as a white powder (1.44 g, 65%). LCMS (ES+) 219 (M+H)⁺.

### Step 2: Methyl 3-benzyloxy-1-phenyl-1H-pyrazole-5-carboxylate

A suspension of methyl 3-hydroxy-1-phenyl-1H-pyrazole-5-carboxylate (106 mg, 0.49 mmol), benzyl bromide (60 μL, 0.51 mmol) and K₂CO₃ (141 mg, 1.02 mmol) in DMF (2 mL) was stirred at 20° C. for 16 h. The crude mixture was used without purification.

### Step 3: N-Hydroxy-3-benzyloxy-1-phenyl-1H-pyrazole-5-carboxamide

The mixture from step 2 was treated with hydroxylamine (50% w/v in water, 0.15 mL, 2.45 mmol) and NaOH (3.75 M in water, 0.26 mL, 0.98 mmol) and stirred at 20° C. for 1.5 h. Purification by preparative HPLC gave the title compound (43 mg, 29% over two steps). LCMS (ES+) 310 (M+H)⁺, RT 3.60 min (Analytical method 2); ¹H NMR δ (ppm) (400 MHz, DMSO-d₆): 11.04 (1H, s), 9.02 (1H, s), 7.79-7.74 (2H, m), 7.56-7.37 (8H, m), 6.37 (1H, s), 5.34 (2H, s).

### Example 14

### N-Hydroxy-1-phenyl-3-pyrrolidin-1-yl-1H-pyrazole-5-carboxamide

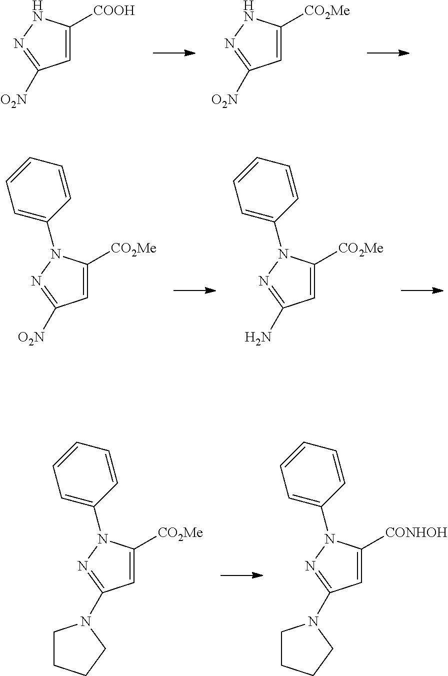

### Step 1: Methyl 3-nitro-1H-pyrazole-5-carboxylate

A stirred solution of 3-nitro-1H-pyrazole-5-carboxylic acid (940 mg, 5.98 mmol) in MeOH (10 mL) at 20° C. was treated cautiously with conc. H₂SO₄ (1 mL). The reaction mixture was heated to 65° C. for 89 h, then concentrated to dryness. The resulting white suspension was triturated with water (10 mL) and the resulting precipitate collected by filtration to yield the title compound as a white solid (718 mg, 70%). LCMS (ES+) 172 (M+H)⁺.

### Step 2: Methyl 3-nitro-1-phenyl-1H-pyrazole-5-carboxylate

Following method C from methyl 3-nitro-1H-pyrazole-5-carboxylate (718 mg, 4.20 mmol) and phenylboronic acid (1.03 g, 8.45 mmol). Purification by silica gel chromatography (gradient elution i-hexane to 100% EtOAc in i-hexane) gave the title compound as a white powder (543 mg, 52%). LCMS (ES+) 248 (M+H)⁺.

### Step 3: Methyl 3-amino-1-phenyl-1H-pyrazole-5-carboxylate

A suspension of methyl 3-nitro-1-phenyl-1H-pyrazole-5-carboxylate (543 mg, 2.20 mmol) in EtOH (11 mL) at 20° C. was treated with iron powder (624 mg, 11.2 mmol) and saturated aqueous NH₄Cl (8 mL) and stirred for 67 h. The temperature was increased to 50° C. and stirring continued for a further 22 h until LCMS indicated complete conversion. The mixture was diluted with EtOAc (400 mL) and filtered through Celite® to remove solids. The filtrate was washed with water (2×200 mL) and brine (100 mL), dried (Na₂SO₄) and concentrated under vacuum. The residue was used immediately without characterisation.

### Step 4: Methyl 1-phenyl-3-pyrrolidin-1-yl-1H-pyrazole-5-carboxylate

A stirred suspension of methyl 3-amino-1-phenyl-1H-pyrazole-5-carboxylate (239 mg, 1.10 mmol), 1,4-diiodobutane (0.14 mL, 1.06 mmol) and Cs₂CO₃ (734 mg, 2.25 mmol) in dry DMF (5.8 mL) was heated at 80° C. under N₂ for 16 h. After cooling to room temperature, the mixture was diluted with CH₂Cl₂ (50 mL), washed with brine (3×30 mL), dried (Na₂SO₄) and concentrated under vacuum. Purification by silica gel chromatography (gradient elution i-hexane to 100% EtOAc in i-hexane) gave the title compound as a colourless liquid (54 mg, 19% over two steps).

### Step 5: N-Hydroxy-1-phenyl-3-pyrrolidin-1-yl-1H-pyrazole-5-carboxamide

Following method A from methyl 1-phenyl-3-pyrrolidin-1-yl-1H-pyrazole-5-carboxylate (54 mg, 0.20 mmol). Purification by preparative HPLC gave the title compound (27 mg, 50%). LCMS (ES+) 273 (M+H)⁺, RT 3.01 min (Analytical method 2); ¹H NMR δ (ppm) (400 MHz, DMSO-d₆): 11.27 (1H, s), 9.30 (1H, s), 7.47-7.40 (4H, m), 7.34-7.28 (1H, m), 6.10 (1H, s), 3.30-3.23 (4H, m), 1.97-1.90 (4H, m).

### Example 15

### N-Hydroxy-4-(2-methylphenyl)-1-phenyl-pyrrole-2-carboxamide

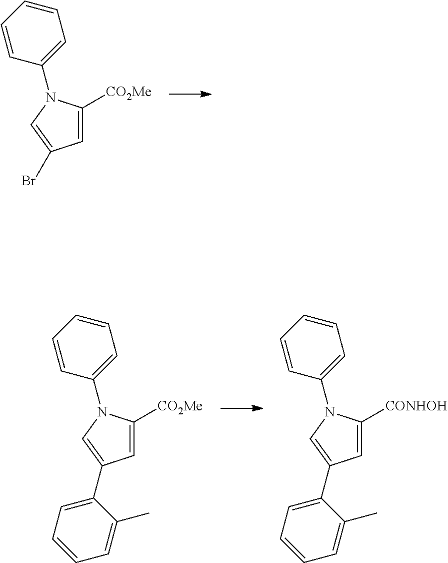

### Step 1: Methyl 4-(2-methylphenyl)-1-phenyl-pyrrole-2-carboxylate

Following method B from intermediate 4 (203 mg, 0.72 mmol) and 2-methylphenylboronic acid (124 mg, 0.91 mmol). Material used immediately without characterization.

### Step 2: N-Hydroxy-4-(2-methylphenyl)-1-phenyl-pyrrole-2-carboxamide

Following method A from methyl 4-(2-methylphenyl)-1-phenyl-pyrrole-2-carboxylate (˜210 mg, 0.72 mmol). Purification by preparative HPLC gave the title compound (23 mg, 11% over two steps). LCMS (ES+) 293 (M+H)⁺, RT 3.80 min (Analytical method 2); ¹H NMR δ (ppm) (400 MHz, DMSO-d₆): 10.99 (1H, s), 9.02 (1H, s), 7.52-7.36 (7H, m), 7.31-7.17 (3H, m), 6.96 (1H, d, J=1.8 Hz), 2.47 (3H, s).

### Example 16

### N-Hydroxy-4-(2-chlorophenyl)-1-phenyl-pyrrole-2-carboxamide

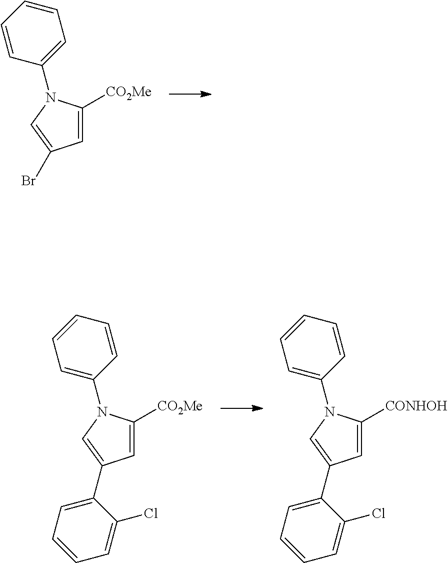

### Step 1: Methyl 4-(2-chlorophenyl)-1-phenyl-pyrrole-2-carboxylate

Following method B from intermediate 4 (299 mg, 1.07 mmol) and 2-chlorophenylboronic acid (229 mg, 1.46 mmol). Material used immediately without characterisation.

### Step 2: N-Hydroxy-4-(2-chlorophenyl)-1-phenyl-pyrrole-2-carboxamide

Following method A from methyl 4-(2-chlorophenyl)-1-phenyl-pyrrole-2-carboxylate (˜334 mg, 1.07 mmol). Purification by preparative HPLC gave the title compound (9 mg, 3% over two steps). LCMS (ES+) 313/315 (M+H)⁺, RT 3.73 min (Analytical method 2); ¹H NMR δ (ppm) (400 MHz, DMSO-d₆): 11.04 (1H, s), 9.04 (1H, s), 7.64 (1H, dd, J=7.7, 1.6 Hz), 7.59-7.37 (8H, m), 7.34-7.28 (1H, m), 7.13 (1H, d, J=1.9 Hz).

### Example 17

### Analysis of Inhibition of HDAC4 with the Compounds

The potency of compounds is quantified by measuring the Histone Deacetylase 4 (HDAC4) catalytic domain enzymatic activity using the fluorogenic substrate, Boc-Lys(Tfa)-AMC. The substrate is deacetylated to Boc-Lys-AMC by HDAC4. Cleavage by trypsin results in the release of the fluorophore AMC from the deacetylated substrate. The fluorescence of the sample is directly related to the histone deacetylase activity in the sample.

Serially Dilute the Compounds.

Serial dilutions of the compounds being tested and control reference compound (1-(5-(3-((4-(1,3,4-oxadiazol-2-yl)phenoxy)methyl)-1,2,4-oxadiazol-5-yl)thiophen-2-yl)-2,2,2-trifluoroethanone) are made by first resuspending the lyophilized compound to a final concentration of 10 mM in 100% dimethyl sulfoxide (DMSO). Stocks of 60 μL aliquots of the 10 mM compound in DMSO are prepared and stored at −20° C. From one stock aliquot of each compound to be tested and the reference compound, a 16-point serial dilution is prepared according to Table 1 using a 125 μL 16-channel Matrix multi-channel pipette (Matrix Technologies Ltd).

TABLE 1
Serial Dilution of Compounds

| | | Concen- | | |
|---|---|---|---|---|
| | | tration | Dilution | |
| Diluted Solutions | Well | (μM) | ratio | Volumes |
| Concentration 1 | A | 10000 | — | 60 μL 10 mM |
| | | | | Test compound/ |
| | | | | reference control |
| Concentration 2 | B | 5000 | 1:2 | 30 μL A + 30 μL DMSO |
| Concentration 3 | C | 2500 | 1:2 | 30 μL B + 30 μL DMSO |
| Concentration 4 | D | 1000 | 1:2.5 | 30 μL C + 45 μL DMSO |
| Concentration 5 | E | 500 | 1:2 | 30 μL D + 30 μL DMSO |
| Concentration 6 | F | 250 | 1:2 | 30 μL E + 30 μL DMSO |
| Concentration 7 | G | 125 | 1:2 | 30 μL F + 30 μL DMSO |
| Concentration 8 | H | 62.5 | 1:2 | 30 μL G + 30 μL DMSO |
| Concentration 9 | I | 31.25 | 1:2 | 30 μL H + 30 μL DMSO |
| Concentration 10 | J | 15.63 | 1:2 | 30 μL I + 30 μL DMSO |
| Concentration 11 | K | 7.81 | 1:2 | 30 μL J + 30 μL DMSO |
| Concentration 12 | L | 3.91 | 1:2 | 30 μL K + 30 μL DMSO |
| Concentration 13 | M | 1.95 | 1:2 | 30 μL L + 30 μL DMSO |
| Concentration 14 | N | 0.98 | 1:2 | 30 μL M + 30 μL DMSO |
| Concentration 15 | O | 0.49 | 1:2 | 30 μL N + 30 μL DMSO |
| Concentration 16 | P | 0.24 | 1:2 | 30 μL O + 30 μL DMSO |

2 μL (200×) of each diluted solution and each control (full activity: 100% DMSO alone or full inhibition 1 mM) is stamped into V-bottomed polypropylene 384-well compound plates using either the Bravo (384-well head from Agilent) or 12.5 μL 16-channel Matrix multi-channel pipette (Matrix Technologies Ltd). Each well with the 200× compound solution is diluted 1:20 by the addition of 38 μL assay buffer+DMSO (10.5% DMSO, 45 mM Tris-HCl, 123 mM NaCl, 2.4 mM KCl, and 0.9 mM MgCl₂ at pH 8.0 and equilibrated to room temperature).

Prepare HDAC4 Catalytic Domain Enzyme (0.2 μg/mL).

The HDAC4 catalytic domain enzyme is human catalytic domain HDAC4 protein (amino acids 648-1032) with a C-terminal 6× histidine tag, produced by BioFocus. A working solution of enzyme is prepared from a 500 μg/mL stock aliquot of HDAC4 catalytic domain (thawed on ice) diluted to 0.2 μg/mL with assay buffer (50 mM Tris-HCl, 137 mM NaCl, 2.7 mM KCl, and 1 mM MgCl₂ at pH 8 and equilibrated to room temperature) just prior to the addition of the enzyme to the assay.

Prepare 5× (50 μM) Boc-Lys(Tfa)-AMC Substrate.

5× (50 μM) substrate is prepared just prior to the addition to the assay. A 1 mM substrate stock is made by diluting a 100 mM Boc-Lys(Tfa)-AMC in DMSO solution 1:100 by adding it drop-wise to assay buffer (equilibrated to room temperature) while vortexing at slow speed to prevent precipitation. The 5× substrate is prepared by diluting the 1 mM substrate solution 1:20 by adding it drop-wise to assay buffer (equilibrated to room temperature) while vortexing at slow speed to prevent precipitation.

Prepare 3× (30 μM) Developer/Stop Solution.

3× (30 μM) Developer/Stop Solution is prepared just prior to addition to the plate by diluting a stock solution of 10 mM reference compound 1:333 in 25 mg/mL trypsin (PAA Laboratories Ltd.) equilibrated to room temperature.

Assay.

5 μL of each solution of 1:20 diluted compound from above is transferred to a clear bottomed, black, 384-well assay plate using the Bravo or the Janus (384-well MDT head from Perkin Elmer). Using a 16-channel Matrix multi-channel pipette, 35 μL of the working solution of HDAC4 catalytic domain enzyme (0.2 μg/mL in assay buffer) is transferred to the assay plate. The assay is then started by adding 10 μL of 5× (50 μM) substrate to the assay plates using either the Bravo, Janus or 16-channel Matrix multi-channel pipette. The assay plate is then shaken for two minutes on an orbital shaker at 900 rpm (rotations per minute). Next the plate is incubated for 15 minutes at 37° C. The reaction is stopped by adding 25 μL of 3× (30 μM) developer/stop solution to the assay plates using either the Bravo, Janus or a 16-channel Matrix multi-channel pipette. Assay plates are then shaken for 5 minutes on an orbital shaker at 1200 rpm. Next, the assay plates are incubated at 37° C. for 1 hour in a tissue culture incubator. Finally, the fluorescence is measured (Excitation: 355 nm, Emission: 460 nm) using PerkinElmer EnVision in top read mode.

### Example 18

### Analysis of Inhibition of HDAC5 with the Compounds

The potency of the compounds is quantified by measuring the Histone Deacetylase 5 (HDAC5) enzymatic activity using the fluorogenic substrate, Boc-Lys(Tfa)-AMC. The substrate is deacetylated to Boc-Lys-AMC by HDAC5. Cleavage by trypsin results in the release of the fluorophore AMC from the deacetylated substrate. The fluorescence of the sample is directly related to the histone deacetylase activity in the sample.

Serially Dilute the Compounds.

Serial dilutions of the compounds and control reference compound (1-(5-(3-((4-(1,3,4-oxadiazol-2-yl)phenoxy)methyl)-1,2,4-oxadiazol-5-yl)thiophen-2-yl)-2,2,2-trifluoroethanone) are made by first resuspending the lyophilized compound to a final concentration of 10 mM in 100% DMSO. Stocks of 60 μL aliquots of the 10 mM compound in DMSO are prepared and stored at −20° C. From one stock aliquot of each compound to be tested and the reference compound, a 16-point serial dilution is prepared according to Table 1 using a 125 μL 16-channel Matrix multi-channel pipette.

2 μL (200×) of each diluted solution and each control (full activity: 100% DMSO alone or full inhibition 1 mM) is stamped into V-bottom polypropylene 384-well compound plates using either Bravo, Janus, or a 12.5 μL 16-channel Matrix multi-channel pipette. Each well with the 2 μL of the 200× stamped compound solution is diluted 1:20 by the addition of 38 μl assay buffer+DMSO (10.5% DMSO, 45 mM Tris-HCl, 123 mM NaCl, 2.4 mM KCl, and 0.9 mM MgCl₂ at pH 8.0 and equilibrated to 37° C.).

Prepare HDAC5 Catalytic Domain Enzyme (0.57 μg/mL).

The HDAC5 catalytic domain enzyme is human HDAC5 catalytic domain (GenBank Accession No. NM_{—}001015053), amino acids 657-1123 with a C-terminal His tag and can be obtained from BPS BioScience. The protein is 51 kDa and is expressed in a baculovirus expression system. A working solution of enzyme is prepared from a 1.65 mg/mL stock aliquot of HDAC5 catalytic domain (thawed on ice) diluted to 0.57 μg/mL with assay buffer (50 mM Tris-HCl, 137 mM NaCl, 2.7 mM KCl, and 1 mM MgCl₂ at pH 8 and equilibrated to 37° C.) just prior to the addition of the enzyme to the assay.

Prepare 5× (40 μM) Boc-Lys(Tfa)-AMC Substrate.

5× (40 μM) substrate is prepared just prior to the addition to the assay. The 5× substrate is prepared by diluting the 100 mM Boc-Lys(Tfa)-AMC in DMSO solution 1:2500 by adding it drop-wise to assay buffer (equilibrated to 37° C.) while vortexing at slow speed to prevent precipitation.

Prepare 3× (30 μM) Developer/Stop Solution.

3× (30 μM) Developer/Stop Solution is prepared just prior to addition to the plate by diluting a stock solution of 10 mM reference compound 1:333 in 25 mg/mL trypsin equilibrated to 37° C.

Assay.

5 μL of each solution of the 1:20 diluted compounds and controls from above is transferred to a clear bottomed, black, 384-well assay plate using the Bravo or Janus. Using a 16-channel Matrix multi-channel pipette, 35 μL of the working solution of the HDAC5 catalytic domain enzyme (0.57 μg/mL in assay buffer) is transferred to the assay plate. The assay is then started by adding 10 μL of 5× (40 μM) substrate to the assay plates using either the Bravo, Janus or 16-channel Matrix multi-channel pipette. The assay plate is then shaken for one minute on an orbital shaker at 900 rpm. Next, the plates are incubated for 15 minutes at 37° C. The reaction is stopped by adding 25 μL of 3× (30 μM) developer/stop solution to the assay plates using either the Bravo, Janus or a 16-channel Matrix multi-channel pipette. Assay plates are then shaken for 2 minutes on an orbital shaker at 900 rpm. Next, the assay plates are incubated at 37° C. for 1 hour in a tissue culture incubator followed by shaking for 1 minute at the maximum rpm on an orbital shaker before reading on the EnVision. Finally, the fluorescence is measured (Excitation: 355 nm, Emission: 460 nm) using PerkinElmer EnVision in top read mode.

### Example 19

### Analysis of Inhibition of HDAC7 with the Compounds

The potency of the compounds is quantified by measuring the Histone Deacetylase 7 (HDAC7) enzymatic activity using the fluorogenic substrate, Boc-Lys(Tfa)-AMC. The substrate is deacetylated to Boc-Lys-AMC by HDAC7. Cleavage by trypsin results in the release of the fluorophore AMC from the deacetylated substrate. The fluorescence of the sample is directly related to the histone deacetylase activity in the sample.

Serially Dilute HDAC Inhibitor Compounds.

Serial dilutions of the compounds to be tested and control reference compound (1-(5-(3-((4-(1,3,4-oxadiazol-2-yl)phenoxy)methyl)-1,2,4-oxadiazol-5-yl)thiophen-2-yl)-2,2,2-trifluoroethanone) are made by first resuspending the lyophilized compound to a final concentration of 10 mM in 100% DMSO. Stocks of 60 μL aliquots of the 10 mM compound in DMSO are prepared and stored at −20° C. From one stock aliquot of each compound to be tested and the reference compound, a 16-point serial dilution is prepared according to Table 1 using a 125 μL 16-channel Matrix multi-channel pipette.

2 μL (200×) of each diluted solution and each control (full activity: 100% DMSO alone or full inhibition 1 mM) is stamped into V-bottom polypropylene 384-well compound plates using either the Bravo, Janus, or a 12.5 μL 16-channel Matrix multi-channel pipette. Each well with the 200× compound solution is diluted 1:20 by the addition of 38 μL assay buffer+DMSO (10.5% DMSO, 45 mM Tris-HCl, 123 mM NaCl, 2.4 mM KCl, and 0.9 mM MgCl₂ at pH 8.0 and equilibrated to 37° C.).

Prepare HDAC7 Enzyme (71 ng/mL).

The HDAC7 enzyme is human HDAC7 (GenBank Accession No. AY302468) amino acids 518-end with a N-terminal Glutathione S-transferase (GST) tag and can be obtained from BPS BioScience. The protein is 78 kDa and is expressed in a baculovirus expression system. A working solution of enzyme is prepared from a 0.5 mg/ml stock aliquot of HDAC7 (thawed on ice) diluted to 71 ng/mL with assay buffer (50 mM Tris-HCl, 137 mM NaCl, 2.7 mM KCl, and 1 mM MgCl₂ at pH 8 and equilibrated to 37° C.) just prior to the addition of enzyme to the assay.

Prepare 5× (50 μM) Boc-Lys(Tfa)-AMC Substrate.

5× (50 μM) substrate is prepared just prior to the addition to the assay. The 5× substrate is prepared by diluting a 100 mM Boc-Lys(Tfa)-AMC in DMSO solution 1:2000 by adding it drop-wise to assay buffer (equilibrated to 37° C.) while vortexing at slow speed to prevent precipitation.

Prepare 3× (30 μM) Developer/Stop Solution.

3× (30 μM) Developer/Stop Solution is prepared just prior to addition to the plate by diluting a stock solution of 10 mM reference compound 1:333 in 25 mg/mL trypsin equilibrated to 37° C.

Assay.

5 μL of each solution of 1:20 diluted compound from above is transferred to a clear bottomed, black, 384-well assay plate using the Bravo or Janus. Using a 16-channel Matrix multi-channel pipette, 35 μL of the working solution of the HDAC7 enzyme (71 ng/mL in assay buffer) is transferred to the assay plate. The assay is then started by adding 10 μL of 5× (50 μM) substrate to the assay plate using either the Bravo, Janus or 16-channel Matrix multi-channel pipette. The assay plate is then shaken for one minute on an orbital shaker at 900 rpm. Next, the plate is incubated for 15 minutes at 37° C. The reaction is then stopped by adding 25 μL of 3× (30 μM) developer/stop solution to the assay plates using either the Bravo, Janus or a 16-channel Matrix multi-channel pipette. The assay plate is then shaken for 2 minutes on an orbital shaker at 900 rpm. Next, the assay plate is incubated at 37° C. for 1 hour in a tissue culture incubator followed by shaking for 1 minute at maximum rpm on an orbital shaker. Finally, the fluorescence is measured (Excitation: 355 nm, Emission: 460 nm) using PerkinElmer EnVision in top read mode.

### Example 20

### Analysis of Inhibition of HDAC9 with the Compounds

The potency of the compounds is quantified by measuring the Histone Deacetylase 9 (HDAC9) enzymatic activity using the fluorogenic substrate, Boc-Lys(Tfa)-AMC. The substrate is deacetylated to Boc-Lys-AMC by HDAC9. Cleavage by trypsin results in the release of the fluorophore AMC from the deacetylated substrate. The fluorescence of the sample is directly related to the histone deacetylase activity in the sample.

Serially Dilute the Compounds.

Serial dilutions of the compounds and control reference compound (1-(5-(3-((4-(1,3,4-oxadiazol-2-yl)phenoxy)methyl)-1,2,4-oxadiazol-5-yl)thiophen-2-yl)-2,2,2-trifluoroethanone) are made by first resuspending the lyophilized compound to a final concentration of 10 mM in 100% DMSO. Stocks of 60 μL aliquots of the 10 mM compound in DMSO are prepared and stored at −20° C. From one stock aliquot of each compound to be tested and the reference compound, a 16-point serial dilution is prepared according to Table 1 using a 125 μL 16-channel Matrix multi-channel pipette.

2 μL (200×) of each diluted solution and each control (full activity: 100% DMSO alone or full inhibition 1 mM) is stamped into V-bottom polypropylene 384-well compound plates using either the Bravo, Janus, or 12.5 μL 16-channel Matrix multi-channel pipette. Each well with the stamped 200× compound solution is diluted 1:20 by the addition of 38 μL assay buffer+DMSO (10.5% DMSO, 45 mM Tris-HCl, 123 mM NaCl, 2.4 mM KCl, and 0.9 mM MgCl₂ at pH 8.0 and equilibrated to 37° C.).

Prepare HDAC9 Enzyme (0.57 μg/mL).

The HDAC9 enzyme is human HDAC9 (GenBank Accession No. NM_{—}178423) amino acids 604-1066 with a C-terminal His tag and can be obtained from BPS BioScience. The protein is 50.7 kDa and is expressed in a baculovirus expression system. A working solution of enzyme is prepared from a 0.5 mg/mL stock aliquot of HDAC9 (thawed on ice) diluted to 0.57 μg/mL with assay buffer (50 mM Tris-HCl, 137 mM NaCl, 2.7 mM KCl, and 1 mM MgCl₂ at pH 8 and equilibrated to 37° C.) just prior to the addition of enzyme to the assay.

Prepare 5× (125 μM) Boc-Lys(Tfa)-AMC Substrate.

5× (125 μM) substrate is prepared just prior to the addition to the assay. The 5× substrate is prepared by diluting a 100 mM Boc-Lys(Tfa)-AMC in DMSO solution 1:800 by adding it drop-wise to assay buffer (equilibrated to 37° C.) while vortexing at slow speed to prevent precipitation.

Prepare 3× (30 μM) Developer/Stop Solution.

3× (30 μM) Developer/Stop Solution is prepared just prior to addition to the plate by diluting a stock solution of 10 mM reference compound 1:333 in 25 mg/mL trypsin equilibrated to 37° C.

Assay.

5 μL of each solution of 1:20 diluted compound from above is transferred to a clear bottomed, black, 384-well assay plate using the Bravo or Janus. Using a 16-channel Matrix multi-channel pipette, 35 μL of the working solution of the HDAC9 enzyme (0.57 μg/mL in assay buffer) is transferred to the assay plate. The assay is then started by adding 10 μL of 5× (125 μM) substrate to the assay plate using either the Bravo, Janus or 16-channel Matrix multi-channel pipette. The assay plate is then shaken for one minute on an orbital shaker at 900 rpm. Next, the plate is incubated for 15 minutes at 37° C. The reaction is stopped by adding 25 μL of 3× developer/stop solution to the assay plates using either the Bravo, Janus or a 16-channel Matrix multi-channel pipette. The assay plate is then shaken for 2 minutes on an orbital shaker at 900 rpm. Next, the assay plate is incubated at 37° C. for 1 hour in a tissue culture incubator followed by shaking for 1 minute at maximum rpm on an orbital shaker before reading on the enVision. Finally, the fluorescence is measured (Excitation: 355 nm, Emission: 460 nm) using PerkinElmer EnVision in top read mode.

### Example 21

### Analysis of Inhibition of Cellular HDAC Activity with the Compounds

The potency of the compounds is quantified by measuring the cellular histone deacetylase enzymatic activity using the fluorogenic substrate, Boc-Lys(Tfa)-AMC. After penetration in Jurkat E6-1 cells, the substrate is deacetylated to Boc-Lys-AMC. After cell lysis and cleavage by trypsin, the fluorophore AMC is released from the deacetylated substrate only. The fluorescence of the sample is directly related to the histone deacetylase activity in the sample.

Jurkat E6.1 Cell Culture and Plating.

Jurkat E6.1 cells are cultured according to standard cell culture protocols in Jurkat E6.1 Growth Media (RPMI without phenol red, 10% FBS, 10 mM HEPES, and 1 mM Sodium Pyruvate). Jurkat E6.1 cells are counted using a Coulter Counter and resuspended in Jurkat E6.1 growth media at a concentration of 75,000 cells/35 μL. 35 μL or 75,000 cells is seeded into Greiner microtitre assay plates. The plates are then incubated at 37° C. and 5% CO₂ while other assay components are being prepared.

Serially Dilute the Compounds.

Serial dilutions of the compounds being tested and control reference compound (1-(5-(3-((4-(1,3,4-oxadiazol-2-yl)phenoxy)methyl)-1,2,4-oxadiazol-5-yl)thiophen-2-yl)-2,2,2-trifluoroethanone) are made by first resuspending the lyophilized compound to a final concentration of 10 mM in 100% DMSO. Stocks of 70 μL aliquots of the 10 mM compound in DMSO are prepared and stored at −20° C. From one stock aliquot of each compound to be tested and the reference compound, a 16-point serial dilution is prepared according to Table 1 using a 125 μL 16-channel Matrix multi-channel pipette.

2 μL (200×) of each diluted solution and each control (full activity: 100% DMSO alone or full inhibition 1 mM) is stamped into V-bottom polypropylene 384-well compound plates using either the Bravo, Janus, or 12.5 μL 16-channel Matrix multi-channel pipette. Each well with the 200× compound solution is diluted 1:20 by the addition of 38 μL Jurkat assay buffer+DMSO (9.5% DMSO, RPMI without phenol red, 0.09% FBS, 9 mM Hepes, and 0.9 mM Sodium Pyruvate equilibrated to room temperature)

Prepare 5× (500 μM) Boc-Lys(Tfa)-AMC Substrate.

5× (500 μM) substrate is prepared just prior to the addition to the assay. The 5× substrate is prepared by diluting a 100 mM Boc-Lys(Tfa)-AMC in DMSO solution 1:200 by adding it drop-wise to Jurkat assay medium (RPMI without phenol red, 0.1% FBS, 10 mM Hepes, and 1 mM Sodium Pyruvate equilibrated to 37° C.) while vortexing at slow speed to prevent precipitation.

Prepare 3× Lysis Buffer.

10 mL of 3× lysis buffer is prepared with 8.8 ml of 3× stock lysis buffer (50 mM Tris-HCl, pH 8.0, 137 mM NaCl, 2.7 mM KCl, 1 mM MgCl₂, 1% Nonidet P40 Substitute equilibrated to room temperature) and 1.2 mL of 3 mg/mL Trypsin equilibrated to room temperature.

Assay.

5 μL of each solution of 1:20 diluted compound from above is transferred to the Greiner microtitre assay plates with 75,000 cells/well using the Bravo. Cells are then incubated for 2 hours at 37° C. and 5% CO₂. The assay is then started by adding 10 μL of 5× (500 μM) substrate to the assay plate using either the Bravo or 16-channel Matrix multi-channel pipette. The cells are then incubated for 3 hours at 37° C. and 5% CO₂. Next, 25 μL of 3× lysis buffer is added to each well using either the 125 μL 16 channel pipette or the Bravo. The assay plate is then incubated overnight (15-16 hours) at 37° C. and 5% CO₂. The following day, the plates are shaken on an orbital shaker for 1 minute at 900 rpm. Finally the top read fluorescence (Excitation: 355 nm, Emission: 460 nm) is measured using PerkinElmer EnVision.

### Example 22

Using the assay protocols described above, the following compounds synthesized by the above synthetic methods were tested.

TABLE 2

| | | Synthetic | HDAC4 | JURKAT TFA |
|---|---|---|---|---|
| compound | Name | Example | IC₅₀ (μM) | IC₅₀ (μM) |
| 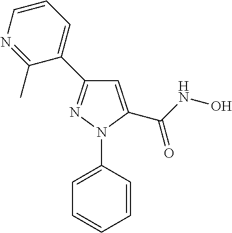 [SMILES: Cc1ncccc1-c1cc(C(=O)NO)n(-c2ccccc2)n1] | N-hydroxy-3-(2- methylpyridin-3- yl)-1-phenyl-1H- pyrazole-5- carboxamide | 9 | 1.4 | 6.9 |
| 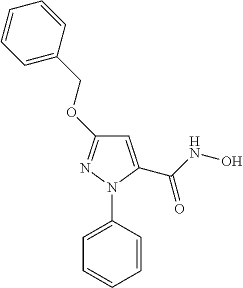 [SMILES: O=C(NO)c1cc(OCc2ccccc2)nn1-c1ccccc1] | 3-(benzyloxy)-N- hydroxy-1-phenyl- 1H-pyrazole-5- carboxamide | 13 | 27.5 | >50 |
| 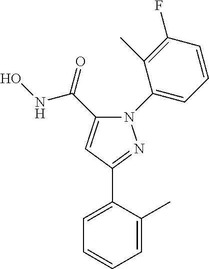 [SMILES: Cc1ccccc1-c1cc(C(=O)NO)n(-c2cccc(F)c2C)n1] | 1-(3-fluoro-2- methylphenyl)-N- hydroxy-3-o-tolyl- 1H-pyrazole-5- carboxamide | 10 | 0.75 | 5.68 |
| 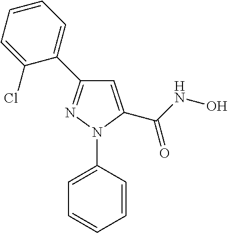 [SMILES: O=C(NO)c1cc(-c2ccccc2Cl)nn1-c1ccccc1] | 3-(2- chlorophenyl)-N- hydroxy-1-phenyl- 1H-pyrazole-5- carboxamide | 5 | 0.38 | 2.82 |
| 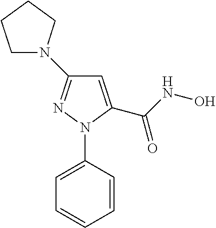 [SMILES: O=C(NO)c1cc(N2CCCC2)nn1-c1ccccc1] | N-hydroxy-1- phenyl-3- (pyrrolidin-1-yl)- 1H-pyrazole-5- carboxamide | 14 | 9.9 | 46.2 |
| 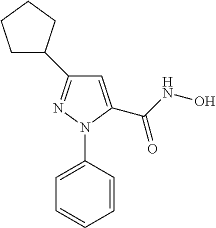 [SMILES: O=C(NO)c1cc(C2CCCC2)nn1-c1ccccc1] | 3-cyclopentyl-N- hydroxy-1-phenyl- 1H-pyrazole-5- carboxamide | 7 | 1.8 | 12.1 |
| 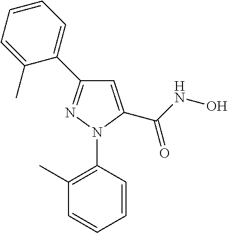 [SMILES: Cc1ccccc1-c1cc(C(=O)NO)n(-c2ccccc2C)n1] | N-hydroxy-1,3- dio-tolyl-1H- pyrazole-5- carboxamide | 12 | 2.4 | 16.0 |
| 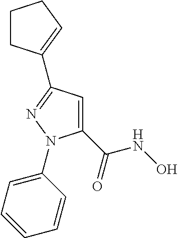 [SMILES: O=C(NO)c1cc(C2=CCCC2)nn1-c1ccccc1] | 3-cyclopentenyl- N-hydroxy-1- phenyl-1H- pyrazole-5- carboxamide | 6 | 2.7 | 20.3 |
| 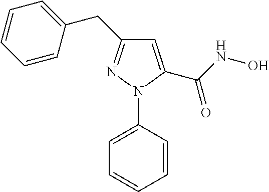 [SMILES: O=C(NO)c1cc(Cc2ccccc2)nn1-c1ccccc1] | 3-benzyl-N- hydroxy-1-phenyl- 1H-pyrazole-5- carboxamide | 4 | 0.96 | 10.2 |
| 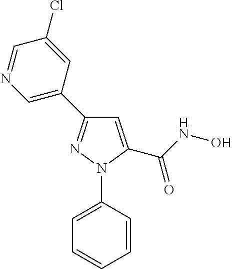 [SMILES: O=C(NO)c1cc(-c2cncc(Cl)c2)nn1-c1ccccc1] | 3-(5- chloropyridin-3- yl)-N-hydroxy-1- phenyl-1H- pyrazole-5- carboxamide | 8 | 4.9 | 41.6 |
| 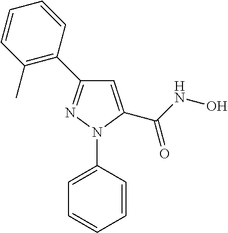 [SMILES: Cc1ccccc1-c1cc(C(=O)NO)n(-c2ccccc2)n1] | N-hydroxy-1- phenyl-3-o-tolyl- 1H-pyrazole-5- carboxamide | 3 | 0.94 | 7.30 |
| 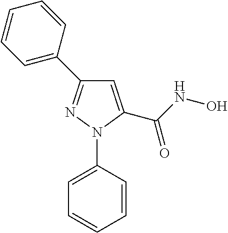 [SMILES: O=C(NO)c1cc(-c2ccccc2)nn1-c1ccccc1] | N-hydroxy-1,3- diphenyl-1H- pyrazole-5- carboxamide | 1 | 10.3 | 24.2 |
| 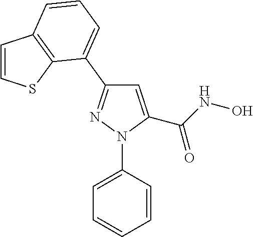 [SMILES: O=C(NO)c1cc(-c2cccc3ccsc23)nn1-c1ccccc1] | 3- (benzo[b]thiophen- 7-yl)-N-hydroxy- 1-phenyl-1H- pyrazole-5- carboxamide | 2 | 0.12 | 2.72 |
| 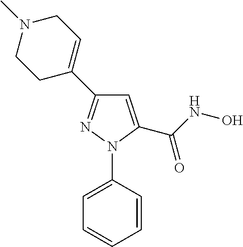 [SMILES: CN1CC=C(c2cc(C(=O)NO)n(-c3ccccc3)n2)CC1] | N-hydroxy-3-(1- methyl-1,2,3,6- tetrahydropyridin- 4-yl)-1-phenyl-1H- pyrazole-5- carboxamide | 11 | 0.24 | 4.07 |
| 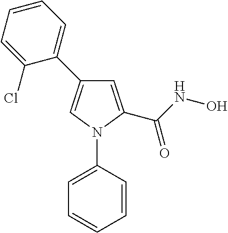 [SMILES: O=C(NO)c1cc(-c2ccccc2Cl)cn1-c1ccccc1] | 4-(2- chlorophenyl)-N- hydroxy-1-phenyl- 1H-pyrrole-2- carboxamide | 16 | 0.71 | 6.17 |
| 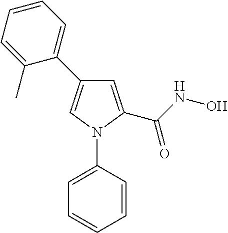 [SMILES: Cc1ccccc1-c1cc(C(=O)NO)n(-c2ccccc2)c1] | N-hydroxy-1- phenyl-4-o-tolyl- 1H-pyrrole-2- carboxamide | 15 | 1.4 | 13.8 |

### Example 23

Using the synthetic methods similar to those described above and the assay protocols described above, the following compounds can be synthesized and tested.

TABLE 3
Additional Compounds
Name

N-hydroxy-1-phenyl-3-(1-(2,2,2-trifluoroethyl)piperidin-4-yl)-1H-pyrazole-5-carboxamide
N-hydroxy-3-(1-isopropylpiperidin-4-yl)-1-phenyl-1H-pyrazole-5-carboxamide
3-(1-cyclopropylpyrrolidin-3-yl)-N-hydroxy-1-phenyl-1H-pyrazole-5-carboxamide
N-hydroxy-1-phenyl-3-(4-(2,2,2-trifluoroethyl)piperazin-1-yl)-1H-pyrazole-5-carboxamide
N-hydroxy-3-(4-isopropylpiperazin-1-yl)-1-phenyl-1H-pyrazole-5-carboxamide
3-(4-cyclopropylpiperazin-1-yl)-N-hydroxy-1-phenyl-1H-pyrazole-5-carboxamide
N-hydroxy-1-phenyl-3-((4-(2,2,2-trifluoroethyl)piperazin-1-yl)methyl)-1H-p yrazole-5-
carboxamide
N-hydroxy-3-(1-(4-isopropylpiperazin-1-yl)cyclopropyl)-1-phenyl-1H-pyrazole-5-carboxamide
3-((4-cyclopropylpiperazin-1-yl)methyl)-N-hydroxy-1-phenyl-1H-pyrazole-5-carboxamide
N-hydroxy-1-phenyl-3-((4-(2,2,2-trifluoroethyl)piperazin-1-yl)methyl)-1H-pyrazole-5-
carboxamide
N-hydroxy-3-(1-(4-isopropylpiperazin-1-yl)cyclopropyl)-1-phenyl-1H-pyrazole-5-carboxamide
3-((4-cyclopropylpiperazin-1-yl)methyl)-N-hydroxy-1-phenyl-1H-pyrazole-5-carboxamide
3-((4-fluorophenyl)(phenyl)methyl)-N-hydroxy-1-phenyl-1H-pyrazole-5-carboxamide
3-((4-fluorophenyl)(phenyl)amino)-N-hydroxy-1-phenyl-1H-pyrazole-5-carboxamide
3-(2,3-dihydrobenzofuran-7-yl)-N-hydroxy-1-phenyl-1H-pyrazole-5-carboxamide
N-hydroxy-3-(4-methyl-3,4-dihydro-2H-benzo[b][1,4]oxazin-8-yl)-1-phenyl-1H-pyrazole-5-
carboxamide
3-(4-chlorobenzo[d]thiazol-5-yl)-N-hydroxy-1-phenyl-1H-pyrazole-5-carboxamide

While some embodiments have been shown and described, various modifications and substitutions may be made thereto without departing from the spirit and scope of the invention. For example, for claim construction purposes, it is not intended that the claims set forth hereinafter be construed in any way narrower than the literal language thereof, and it is thus not intended that exemplary embodiments from the specification be read into the claims. Accordingly, it is to be understood that the present invention has been described by way of illustration and not limitations on the scope of the claims.

1. A compound of Formula I: 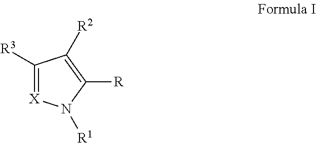
or a pharmaceutically acceptable salt thereof, wherein
X is CR⁴ or N;
R is chosen from —C(O)NH(OH) and —N(OH)C(O)R⁷;
R¹ is optionally substituted aryl or optionally substituted heteroaryl;
R² is chosen from hydrogen, C₁-C₄ alkyl, halo, C₁-C₄ haloalkyl, and nitrile;
R³ is chosen from —OR⁵, NR⁵R⁶, optionally substituted alkyl, optionally substituted aralkyl, optionally substituted aryl, optionally substituted heteroaryl, optionally substituted heterocycloalkyl, optionally substituted heterocycloalkenyl, optionally substituted cycloalkenyl and optionally substituted cycloalkyl;
R⁴ is chosen from hydrogen, halo, C₁-C₄ alkyl or C₁-C₄ haloalkyl;
R⁵ and R⁶ are independently chosen from hydrogen, optionally substituted C₁-C₄ alkyl, optionally substituted C₁-C₄ haloalkyl, optionally substituted aryl, optionally substituted heteroaryl, optionally substituted heterocycloalkyl, optionally substituted cycloalkyl, optionally substituted aralkyl and optionally substituted heteroaralkyl; or R⁵ and R⁶, together with the nitrogen atom to which they are attached, form an optionally substituted heterocycloalkyl; and
R⁷ is chosen from hydrogen, C₁-C₄ alkyl and C₁-C₄ haloalkyl.

2. The compound of claim 1, or a pharmaceutically acceptable salt thereof, wherein
R¹ is aryl or heteroaryl, each of which is optionally substituted with 1 to 3 substituents independently chosen from halo, C₁-C₄ alkyl, C₁-C₄ haloalkyl, hydroxyl, alkoxy, and nitrile;
R³ is chosen from —OR⁵, —NR⁵R⁶, alkyl, aralkyl, aryl, heteroaryl, heterocycloalkyl, heterocycloalkenyl, cycloalkenyl and cycloalkyl, wherein the aryl, heteroaryl, heterocycloalkyl, heterocycloalkenyl, cycloalkenyl or cycloalkyl is optionally substituted with 1 to 3 substituents independently chosen from halo, C₁-C₄ alkyl, C₁-C₄ haloalkyl, hydroxyl, alkoxy, nitrile, heteroaryl, phenyl, heterocycloalkyl, cycloalkyl, aralkyl and heteroaralkyl; and
R⁵ and R⁶ are independently chosen from hydrogen, C₁-C₄ alkyl, C₁-C₄ haloalkyl, heteroaryl, heterocycloalkyl, cycloalkyl, aryl, aralkyl and heteroaralkyl, wherein the heteroaryl, heterocycloalkyl, cycloalkyl, aryl, aralkyl or heteroaralkyl is optionally substituted with 1 to 3 substituents independently chosen from halo, C₁-C₄ alkyl, C₁-C₄ haloalkyl, hydroxyl, alkoxy, and nitrile; or R⁵ and R⁶, together with the nitrogen atom to which they are attached, form an optionally substituted heterocycloalkyl comprising one or two heteroatoms.

3. The compound of claim 1, or a pharmaceutically acceptable salt thereof, wherein R is —C(O)NH(OH).

4. The compound of claim 1, or a pharmaceutically acceptable salt thereof, wherein R is —N(OH)C(O)R⁷.

5. The compound of claim 4, or a pharmaceutically acceptable salt thereof, wherein R⁷ is chosen from hydrogen and C₁-C₄ alkyl.

6. The compound of claim 5, or a pharmaceutically acceptable salt thereof, wherein R⁷ is C₁-C₄ alkyl.

7. The compound of claim 1, or a pharmaceutically acceptable salt thereof, wherein X is CR⁴.

8. The compound of claim 1, or a pharmaceutically acceptable salt thereof, wherein R⁴ is hydrogen or C₁-C₄ alkyl.

9. The compound of claim 8, or a pharmaceutically acceptable salt thereof, wherein R⁴ is hydrogen.

10. The compound of claim 1, or a pharmaceutically acceptable salt thereof, wherein X is N.

11. The compound of claim 1, or a pharmaceutically acceptable salt thereof, wherein R¹ is aryl optionally substituted with 1 to 3 substituents independently chosen from halo, C₁-C₄ alkyl, C₁-C₄ haloalkyl, hydroxyl, alkoxy, and nitrile.

12. The compound of claim 11, or a pharmaceutically acceptable salt thereof, wherein R¹ is phenyl optionally substituted with 1 or 2 substituents independently chosen from C₁-C₄ alkyl and halo.

13. The compound of claim 12, or a pharmaceutically acceptable salt thereof, wherein R¹ is phenyl.

14. The compound of claim 1, or a pharmaceutically acceptable salt thereof, wherein R² is chosen from hydrogen, C₁-C₄ alkyl, halo, and C₁-C₄ haloalkyl.

15. The compound of claim 14, or a pharmaceutically acceptable salt thereof, wherein R² is hydrogen.

16. The compound of claim 1, or a pharmaceutically acceptable salt thereof, wherein R³ is —OR⁵.

17. The compound of claim 16, or a pharmaceutically acceptable salt thereof, wherein R⁵ is hydrogen, C₁-C₄ alkyl, or aralkyl.

18. The compound of claim 1, or a pharmaceutically acceptable salt thereof, wherein R³ is —NR⁵R⁶.

19. The compound of claim 18, or a pharmaceutically acceptable salt thereof, wherein R⁵ and R⁶, together with the nitrogen atom to which they are attached, form an optionally substituted heterocycloalkyl comprising one or two heteroatoms.

20. The compound of claim 19, or a pharmaceutically acceptable salt thereof, wherein R⁵ and R⁶, together with the nitrogen atom to which they are attached, form a heterocycloalkyl chosen from pyrrolidin-1-yl, piperazin-1-yl, piperidine-1-yl, and morpholin-4-yl, each of which is optionally substituted with 1 or 2 substituents independently chosen from C₁-C₄ alkyl, C₁-C₄ haloalkyl, cycloalkyl, halo, and phenyl, wherein the phenyl is optionally substituted with 1 or 2 substituents chosen from C₁-C₄ alkyl, C₁-C₄ haloalkyl and halo.

21. The compound of claim 18, or a pharmaceutically acceptable salt thereof, wherein R⁵ is phenyl optionally substituted with 1 or 2 substituents chosen from C₁-C₄ alkyl, C₁-C₄ haloalkyl and halo.

22. The compound of claim 18, or a pharmaceutically acceptable salt thereof, wherein R⁶ is phenyl optionally substituted with 1 or 2 substituents chosen from C₁-C₄ alkyl, C₁-C₄ haloalkyl and halo.

23. The compound of claim 1, or a pharmaceutically acceptable salt thereof, wherein R³ is optionally substituted aryl.

24. The compound of claim 23, or a pharmaceutically acceptable salt thereof, wherein R³ is aryl optionally substituted with 1 to 3 substituents independently chosen from halo, C₁-C₄ alkyl, C₁-C₄ haloalkyl, hydroxyl, alkoxy, C₃-C₆ cycloalkyl and nitrile.

25. The compound of claim 24, or a pharmaceutically acceptable salt thereof, wherein R³ is phenyl, 2,3-dihydrobenzofuran-7-yl, benzo[d][1,3]dioxol-4-yl, chroman-8-yl, 2,3-dihydrobenzo[b][1,4]dioxin-5-yl, and 3,4-dihydro-2H-benzo[b][1,4]oxazin-8-yl, each of which is optionally substituted with 1 to 3 substituents independently chosen from halo, C₁-C₄ alkyl, C₁-C₄ haloalkyl, hydroxyl, alkoxy, C₃-C₆ cycloalkyl and nitrile.

26. The compound of claim 1, or a pharmaceutically acceptable salt thereof, wherein R³ is optionally substituted heteroaryl.

27. The compound of claim 26, or a pharmaceutically acceptable salt thereof, wherein R³ is heteroaryl optionally substituted with 1 to 3 substituents independently chosen from halo, C₁-C₄ alkyl, C₁-C₄ haloalkyl, hydroxyl, alkoxy, C₃-C₆ cycloalkyl and nitrile.

28. The compound of claim 27, or a pharmaceutically acceptable salt thereof, wherein R³ is pyridin-3-yl, benzofuran-7-yl, benzo[b]thiophen-7-yl, and benzo[d]thiazol-4-yl, each of which is optionally substituted with 1 to 3 substituents independently chosen from halo, C₁-C₄ alkyl, C₁-C₄ haloalkyl, hydroxyl, alkoxy, C₃-C₆ cycloalkyl and nitrile.

29. The compound of claim 1, or a pharmaceutically acceptable salt thereof, wherein R³ is optionally substituted cycloalkyl or optionally substituted cycloalkenyl.

30. The compound of claim 29, or a pharmaceutically acceptable salt thereof, wherein R³ is cycloalkyl or cycloalkenyl, each of which is optionally substituted with 1 to 3 substituents independently chosen from halo, C₁-C₄ alkyl, C₁-C₄ haloalkyl, hydroxyl, alkoxy, C₃-C₆ cycloalkyl, heteroaryl and nitrile.

31. The compound of claim 30, or a pharmaceutically acceptable salt thereof, wherein R³ is chosen from cyclopentyl, cyclohexyl, cyclopentenyl and cyclohexenyl, each of which is optionally substituted with 1 to 3 substituents independently chosen from halo, C₁-C₄ alkyl, C₁-C₄ haloalkyl, hydroxyl, alkoxy, C₃-C₆ cycloalkyl, heteroaryl and nitrile.

32. The compound of claim 1, or a pharmaceutically acceptable salt thereof, wherein R³ is optionally substituted heterocycloalkyl or optionally substituted heterocycloalkenyl.

33. The compound of claim 32, or a pharmaceutically acceptable salt thereof, wherein R³ is heterocycloalkyl or heterocycloalkenyl, each of which is optionally substituted with 1 to 3 substituents independently chosen from halo, C₁-C₄ alkyl, C₁-C₄ haloalkyl, hydroxyl, alkoxy, C₃-C₆ cycloalkyl, aryl and heteroaryl and nitrile.

34. The compound of claim 33, or a pharmaceutically acceptable salt thereof, wherein R³ is piperidin-4-yl or 1,2,3,6-tetrahydropyridin-4-yl, each of which is optionally substituted with 1 to 3 substituents independently chosen from halo, C₁-C₄ alkyl, C₁-C₄ haloalkyl, hydroxyl, alkoxy, C₃-C₆ cycloalkyl and nitrile.

35. The compound of claim 1, or a pharmaceutically acceptable salt thereof, wherein R³ is optionally substituted alkyl.

36. The compound of claim 35, or a pharmaceutically acceptable salt thereof, wherein R³ is alkyl optionally substituted with 1 to 3 substituents independently chosen from halo, C₁-C₄ alkyl, C₁-C₄ haloalkyl, hydroxyl, alkoxy, C₃-C₆ cycloalkyl, optionally substituted heterocycloalkyl, optionally substituted heteroaryl, and nitrile, wherein the heterocycloalkyl and heteroaryl, and groups are optionally substituted with 1 to 3 substituents independently chosen from halo, C₁-C₄ alkyl, C₁-C₄ haloalkyl, hydroxyl, alkoxy, C₃-C₆ cycloalkyl, and nitrile.

37. The compound of claim 36, or a pharmaceutically acceptable salt thereof, wherein R³ is C₁-C₄ alkyl optionally substituted with 1 to 3 substituents independently chosen from halo, C₁-C₄ alkyl, C₁-C₄ haloalkyl, hydroxyl, alkoxy, C₃-C₆ cycloalkyl, optionally substituted heterocycloalkyl, optionally substituted heteroaryl, and nitrile, wherein the heterocycloalkyl and heteroaryl groups are optionally substituted with 1 to 3 substituents independently chosen from halo, C₁-C₄ alkyl, C₁-C₄ haloalkyl, hydroxyl, alkoxy, C₃-C₆ cycloalkyl, and nitrile.

38. The compound of claim 1, or a pharmaceutically acceptable salt thereof, wherein R³ is optionally substituted aralkyl.

39. The compound of claim 38, or a pharmaceutically acceptable salt thereof, wherein R³ is aralkyl optionally substituted with 1 to 3 substituents independently chosen from halo, C₁-C₄ alkyl, C₁-C₄ haloalkyl, hydroxyl, alkoxy, C₃-C₆ cycloalkyl, optionally substituted heterocycloalkyl, optionally substituted heteroaryl, and nitrile, wherein the heterocycloalkyl, and heteroaryl groups are optionally substituted with 1 to 3 substituents independently chosen from halo, C₁-C₄ alkyl, C₁-C₄ haloalkyl, hydroxyl, alkoxy, C₃-C₆ cycloalkyl, and nitrile.

40. The compound of claim 1, or a pharmaceutically acceptable salt thereof, wherein R³ is chosen from methylpyridyl, chloropyridyl, phenyl, methylphenyl, chlorophenyl, benzoxy, pyrrolidinyl, cyclopentyl, cyclopentenyl, benzyl, benzothiophenyl, 1-methyl-1,2,3,6-tetrahydropyridin-4-yl, 1-(2,2,2-trifluoroethyl)piperidin-4-yl, 1-isopropylpiperidin-4-yl, 1-cyclopropylpyrrolidin-3-yl, 4-(2,2,2-trifluoroethyl)piperazin-1-yl, 4-isopropylpiperazin-1-yl, 4-cyclopropylpiperazin-1-yl, 4-(2,2,2-trifluoroethyl)piperazin-1-yl)methyl, 1-(4-isopropylpiperazin-1-yl)cyclopropyl, 4-cyclopropylpiperazin-1-yl)methyl, (4-(2,2,2-trifluoroethyl)piperazin-1-yl)methyl, (4-fluorophenyl)(phenyl)methyl, (4-fluorophenyl)(phenyl)amino, 2,3-dihydrobenzofuran-7-yl, 4-methyl-3,4-dihydro-2H-benzo[b][1,4]oxazin-8-yl, and 4-chlorobenzo[d]thiazol-5-yl.

41. A compound chosen from
N-hydroxy-3-(2-methylpyridin-3-yl)-1-phenyl-1H-pyrazole-5-carboxamide,
3-(benzyloxy)-N-hydroxy-1-phenyl-1H-pyrazole-5-carboxamide,
1-(3-fluoro-2-methylphenyl)-N-hydroxy-3-o-tolyl-1H-pyrazole-5-carboxamide,
3-(2-chlorophenyl)-N-hydroxy-1-phenyl-1H-pyrazole-5-carboxamide,
N-hydroxy-1-phenyl-3-(pyrrolidin-1-yl)-1H-pyrazole-5-carboxamide,
3-cyclopentyl-N-hydroxy-1-phenyl-1H-pyrazole-5-carboxamide,
N-hydroxy-1,3-di-o-tolyl-1H-pyrazole-5-carboxamide,
3-cyclopentenyl-N-hydroxy-1-phenyl-1H-pyrazole-5-carboxamide,
3-benzyl-N-hydroxy-1-phenyl-1H-pyrazole-5-carboxamide,
3-(5-chloropyridin-3-yl)-N-hydroxy-1-phenyl-1H-pyrazole-5-carboxamide,
N-hydroxy-1-phenyl-3-o-tolyl-1H-pyrazole-5-carboxamide,
N-hydroxy-1,3-diphenyl-1H-pyrazole-5-carboxamide,
3-(benzo[b]thiophen-7-yl)-N-hydroxy-1-phenyl-1H-pyrazole-5-carboxamide,
N-hydroxy-3-(1-methyl-1,2,3,6-tetrahydropyridin-4-yl)-1-phenyl-1H-pyrazole-5-carboxamide,
4-(2-chlorophenyl)-N-hydroxy-1-phenyl-1H-pyrrole-2-carboxamide, and
N-hydroxy-1-phenyl-4-o-tolyl-1H-pyrrole-2-carboxamide;
or a pharmaceutically acceptable salt thereof.

42. A compound chosen from
N-hydroxy-1-phenyl-3-(1-(2,2,2-trifluoroethyl)piperidin-4-yl)-1H-pyrazole-5-carboxamide,
N-hydroxy-3-(1-isopropylpiperidin-4-yl)-1-phenyl-1H-pyrazole-5-carboxamide,
3-(1-cyclopropylpyrrolidin-3-yl)-N-hydroxy-1-phenyl-1H-pyrazole-5-carboxamide,
N-hydroxy-1-phenyl-3-(4-(2,2,2-trifluoroethyl)piperazin-1-yl)-1H-pyrazole-5-carboxamide,
N-hydroxy-3-(4-isopropylpiperazin-1-yl)-1-phenyl-1H-pyrazole-5-carboxamide,
3-(4-cyclopropylpiperazin-1-yl)-N-hydroxy-1-phenyl-1H-pyrazole-5-carboxamide,
N-hydroxy-1-phenyl-3-((4-(2,2,2-trifluoroethyl)piperazin-1-yl)methyl)-1H-pyrazole-5-carboxamide,
N-hydroxy-3-(1-(4-isopropylpiperazin-1-yl)cyclopropyl)-1-phenyl-1H-pyrazole-5-carboxamide,
3-((4-cyclopropylpiperazin-1-yl)methyl)-N-hydroxy-1-phenyl-1H-pyrazole-5-carboxamide,
3-((4-fluorophenyl)(phenyl)methyl)-N-hydroxy-1-phenyl-1H-pyrazole-5-carboxamide,
3-((4-fluorophenyl)(phenyl)amino)-N-hydroxy-1-phenyl-1H-pyrazole-5-carboxamide,
3-(2,3-dihydrobenzofuran-7-yl)-N-hydroxy-1-phenyl-1H-pyrazole-5-carboxamide,
N-hydroxy-3-(4-methyl-3,4-dihydro-2H-benzo[b][1,4]oxazin-8-yl)-1-phenyl-1H-pyrazole-5-carboxamide, and
3-(4-chlorobenzo[d]thiazol-5-yl)-N-hydroxy-1-phenyl-1H-pyrazole-5-carboxamide;
or a pharmaceutically acceptable salt thereof.

43. A pharmaceutically acceptable composition comprising a pharmaceutically acceptable carrier and at least one compound of claim 1, or a pharmaceutically acceptable salt thereof.

44. A pharmaceutical composition of claim 43, wherein the composition is formulated in a form chosen from tablets, capsules, powders, liquids, suspensions, suppositories, and aerosols.

45. A method for treating a condition or disorder mediated by at least one histone deacetylase in a patient in need of such a treatment which method comprises administering to the patient a therapeutically effective amount of at least one compound of claim 1, or a pharmaceutically acceptable salt thereof.

46. A method for treating a condition or disorder responsive to inhibition of at least one histone deacetylase in a patient in need of such a treatment which method comprises administering to the patient an effective amount of at least one compound of claim 1, or a pharmaceutically acceptable salt thereof.

47. A method for inhibiting at least one histone deacetylase which method comprises contacting the histone deacetylase with an effective amount of at least one compound of claim 1, or a pharmaceutically acceptable salt thereof.

48. The method of claim 45, wherein the at least one histone deacetylase is HDAC-4.

49. The method of claim 45, wherein said condition or disorder involves a neurodegenerative pathology.

50. The method of claim 45, wherein the condition or disorder is Huntington's disease.
